(19) United States
(12) Patent Application Publication
(10) Pub. No.: US 2025/0127702 A1
(43) Pub. Date: Apr. 24, 2025

(54) SUNSCREEN COMPOSITION WITH CRYSTALLINE ORGANIC SUNSCREEN FILTERS

(71) Applicant: Edgewell Personal Care Brands, LLC, Chesterfield, MO (US)

(72) Inventors: David Gee, New Smyrna Beach, FL (US); James Grant, New Providence, NJ (US); Cassandra Wagers, Edgewater, FL (US)

(21) Appl. No.: 18/694,451

(22) PCT Filed: Sep. 23, 2022
(86) PCT No.: PCT/US2022/076899

§ 371 Date: Mar. 22, 2024

Related U.S. Application Data
(60) Provisional application No. 63/247,488, filed on Sep. 23, 2021.

(51) Int. Cl. A61K 8/49; A61K 8/35; A61Q 17/04

(52) U.S. Cl. CPC A61K 8/4966; A61K 8/35; A61Q 17/04; A61K 2800/30

(57) ABSTRACT

A sunscreen composition includes a solvent and a sunscreen filter dissolved in the solvent. The solvent may include an ester. The ester may include an ester of salicylic acid, an ester of adipic acid, an ester of maleic acid, an ester of neopentanoic acid, an ester of C₈-C₁₈ fatty acids, or combinations thereof. The sunscreen filter may include a crystalline ultraviolet A (UVA) filter and/or a crystalline UVB filter. The sunscreen filter may include butyl methoxydibenzoylmethane, diethylhexyl butamido triazone, diethylamino hydroxybenzoyl hexyl benzoate, bis-ethylhexyloxyphenol methoxyphenyl triazine, ethylhexyl triazone, or combinations thereof. The sunscreen composition may exclude ethylhexyl benzoate, phenethyl benzoate, C₁₂-C₁₅ alkyl benzoate, or combinations thereof. The sunscreen composition may exclude benzophenone-3, octyl methoxycinnamate, 2-ethylhexyl-2-cyano-3,3-diphenylacrylate, 3,3,5-trimethylcyclohexyl salicylate, 2-ethylhexyl salicylate, or combinations thereof.

### CROSS-REFERENCE TO RELATED APPLICATIONS

This application claims priority to U.S. Provisional Patent Application No. 63/247,488, filed on Sep. 23, 2021, the contents of which is incorporated herein by reference in its entirety.

### TECHNICAL FIELD

Embodiments relate to a sunscreen composition including a solvent and one or more sunscreen filters. A solvent may include an ester. A mixture of sunscreen filters may include a crystalline ultraviolet A filter and/or a crystalline ultraviolet B filter.

### BACKGROUND

Sunscreen compositions may include organic sunscreen filters capable of absorbing ultraviolet (UV) radiation. Organic sunscreen filters, however, may be crystalline at room temperature and are generally used at relatively high levels. Thus, the solubility of organic crystalline sunscreen filters in a homogeneous and/or stable manner is a crucial attribute to provide sufficiently high or reliable photoprotection in sunscreen compositions.

Some oily or ester ingredients have good dissolving power for crystalline sunscreen filters. Such oily or ester ingredients may, however, still have insufficient power to dissolve relatively large amounts of crystalline sunscreen filters conventionally used. While the levels of such oily or ester ingredients may be increased to keep the crystalline sunscreen filters from precipitating out of solution (thereby keeping the sunscreen composition effective), improved solubility may come at the expense of sensory attributes that cause a negative impact on consumer perception. In addition, compositions containing a high solvent load will tend to migrate on a user's skin and significantly reduce sun protection factor (SPF) efficacy (according to Beer's Law). Thus, migration also continues to be a major factor in the lack of commercial anhydrous alcohol-based or aqueous emulsion compositions having only crystalline sunscreen filters.

Meanwhile, certain ester ingredients and organic liquid sunscreen filters that solvate crystalline sunscreen filters are increasingly raising concerns. Omitting such solubilizing ingredients to address such concerns, however, dampens the dissolving power for crystalline sunscreen filters and may drive the need for even higher levels of crystalline sunscreen filters further exacerbating technical challenges related to solubility. Thus, there is a considerable need for sunscreen compositions having crystalline sunscreen filters solubilized in a homogeneous and/or stable manner to provide sufficiently high or reliable photoprotection.

### SUMMARY

Embodiments relate to a sunscreen composition including a solvent and a mixture of sunscreen filters, preferably a crystalline ultraviolet A (UVA) filter and a crystalline ultraviolet B (UVB) filter, wherein the solubility of the mixture, and in particular of the crystalline UVA filter, is enhanced. Thus, the mixture of crystalline sunscreen filters may be dispersed in a homogeneous and stable manner such that precipitation out of solution is minimized or prevented. In this regard, sufficiently high and/or reliable photoprotection may be provided.

Embodiments relate to a sunscreen composition including a solvent and one or more sunscreen filters dissolved in the solvent. The solvent may include an ester. A mixture of sunscreen filters may include a crystalline UVA filter and/or a crystalline UVB filter. A ratio of the crystalline UVA filter to the crystalline UVB filter may be about 3:1 to about 1:5. For example, the ratio by weight of the crystalline UVA filter to the crystalline UVB filter may be 3.6:0.8, 0.8:6, 3:1, 2.5:1, 2:1, 1.5:1. 1:1, 1:1.5, 1:2, 1:2.5, 1:3, 1:3.5, 1:4, 1:4.5, 1:5, 1:5.5, 1:6, including every number and/or fraction there between.

In any of the preceding embodiments, the crystalline UVA filter may include butyl methoxydibenzoylmethane and may exclude diethylamino hydroxybenzoyl hexyl benzoate.

In any of the preceding embodiments, the crystalline UVB filter may include diethylhexyl butamido triazone. For example, the ratio of butyl methoxydibenzoylmethane to diethylhexyl butamido triazone may be 1.25:1 to 1:1.25. For example, butyl methoxydibenzoylmethane may be present in an amount of about 4 wt. % to about 5 wt. %, based on the total weight of the sunscreen composition, and diethylhexyl butamido triazone may be present in an amount of about 4 wt. % to about 6 wt. %, based on the total weight of the sunscreen composition.

In any of the preceding embodiments, the crystalline UVB filter may include ethylhexyl triazone. For example, diethylhexyl butamido triazone may be present in an amount ≥80% of the amount of butyl methoxydibenzoylmethane present in the sunscreen composition. Additionally or alternatively, diethylhexyl butamido triazone may be present in an amount ≥ 80% of the total amount of all crystalline UVB sunscreen filters present in the sunscreen composition. Additionally or alternatively, ethylhexyl triazone may be present in an amount less than the amount of diethylhexyl butamido triazone present.

In any of the preceding embodiments, the crystalline UVA filter may include diethylamino hydroxybenzoyl hexyl benzoate and the crystalline UVB filter may include diethylhexyl butamido triazone, ethylhexyl triazone, or combinations thereof. For example, the ratio of diethylamino hydroxybenzoyl hexyl benzoate to diethylhexyl butamido triazone or to ethylhexyl triazone may be from 1:1 to 1:5. For example, diethylamino hydroxybenzoyl hexyl benzoate may be present in an amount of about 1 wt. % to about 5 wt. %, based on the total weight of the sunscreen composition, and diethylhexyl butamido triazone may be present in an amount of about 1 wt. % to about 10 wt. %, based on the total weight of the sunscreen composition. For example, diethylamino hydroxybenzoyl hexyl benzoate may be present in an amount of about 1 wt. % to about 5 wt. %, based on the total weight of the sunscreen composition, and ethylhexyl triazone may be present in an amount of about 1 wt. % to about 5 wt. %, based on the total weight of the sunscreen composition. In addition, the crystalline UVA filter may further include butyl methoxydibenzoylmethane.

In any of the preceding embodiments, the mixture of sunscreen filters may include a crystalline UVA/UVB filter. For example, the crystalline UVA/UVB filter may include bis-ethylhexyloxyphenol methoxyphenyl triazine. For example, bis-ethylhexyloxyphenol methoxyphenyl triazine may be present in an amount up to about 3 wt. %, based on the total weight of the composition.

In any of the preceding embodiments, the solvent may include an ester of salicylic acid, an ester of adipic acid, an ester of maleic acid, an ester of neopentanoic acid, an ester of C₈-C₁₈ fatty acids, or combinations thereof. For example, the solvent may include butyloctyl salicylate, diisobutyl adipate, dibutyl adipate, diisopropyl adipate, diethylhexyl adipate, diethylhexyl maleate, isostearyl neopentanoate, butyl oleate, decyl oleate, caprylic/capric triglyceride, isopropyl myristate, octyl palmitate, coco caprylate, or combinations thereof.

In any of the preceding embodiments, the solvent may include butyloctyl salicylate, dibutyl adipate, diisobutyl adipate, and caprylic/capric triglyceride. For example, butyloctyl salicylate may be present in an amount of 4 wt. % to 9 wt. %, dibutyl adipate may be present in an amount of 4 wt. % to 13 wt. %, diisobutyl adipate may be present in an amount of 2 wt. % to 20 wt. %, and caprylic/capric triglyceride may be present in an amount of about 5 wt. % to about 10 wt. %, based on the total weight of the composition.

In any of the preceding embodiments, the solvent may include one or more adipates present in a total amount of about 8 wt. % to about 10 wt. %, caprylic/capric triglyceride present in an amount of about 8 wt. % to about 10 wt. %, and butyl octyl salicylate in an amount of 0 wt. % to about 5.0 wt. %. For example, the ratio of adipates to caprylic/capric triglyceride may be about 1:1. Additionally or alternatively, the solvent may exclude diisobutyl adipate and may include butyloctyl salicylate. Additionally or alternatively, the solvent may exclude caprylic/capric triglyceride and may include butyl oleate, dimethyl isosorbide, diethylhexyl adipate, isostearyl neopentanoate, decyl oleate, diethylhexyl maleate, coco caprylate, or combinations thereof. Additionally or alternatively, the solvent may exclude one or more of dibutyl adipate or coco caprylate and may include butyl oleate, dimethyl isosorbide, diethylhexyl adipate, isostearyl neopentonate, decyl oleate, or combinations thereof. Additionally or alternatively, the solvent may include dibutyl adipate and one of diisobutyl adipate or diisopropyl adipate, for example in a ratio of 1:1, and may exclude isopropyl myristate, caprylic/capric triglyceride, or diethylhexyl maleate. Additionally or alternatively, the solvent may exclude dibutyl adipate and caprylic/capric triglyceride and may include dibutyl adipate, for example in an amount greater than zero and ≤20 wt. %, based on the total weight of the composition. Additionally or alternatively, the solvent may include butyloctyl salicylate, dibutyl adipate, caprylic/capric triglyceride, and diethylhexyl maleate. Additionally or alternatively, the solvent may exclude caprylic/capric triglyceride and may include butyloctyl salicylate, dibutyl adipate, diethylhexyl maleate, and isopropyl myristate. Additionally or alternatively, the solvent may exclude butyloctyl salicylate and may include dibutyl adipate, caprylic/capric triglyceride, and diethyl hexyl maleate. Additionally or alternatively, the solvent may include dimethyl isosorbide.

In any of the preceding embodiments, the sunscreen composition may exclude ethylhexyl benzoate, phenethyl benzoate, C₁₂-C₁₅ alkyl benzoate, or combinations thereof.

In any of the preceding embodiments, the solvent may be present in a total amount of about 25.0 wt. % to about 40.0 wt. %, based on the total weight of the composition.

In any of the preceding embodiments, the sunscreen composition may be in the form of a gel, a lotion, a cream, a spray, a mist, a stick, a mousse, a foam, an ointment, a milk, a base, a concentrate, a mist, or combinations thereof. For example, the sunscreen composition may be an aqueous emulsion and the solvent may be a part of an oil phase of the aqueous emulsion. For example, the sunscreen composition may be a substantially anhydrous composition and may include a C₂-C₄ alcohol present in an amount of about 30 wt. % to about 70 wt. %, based on the total weight of the composition.

In any of the preceding embodiments, the sunscreen composition may include a fatty alcohol. For example, the fatty alcohol may include behenyl alcohol. For example, behenyl alcohol may be present in an amount of about 0.1 wt. % to about 1.0 wt. %, based on the total weight of the composition. Additionally or alternatively, the sunscreen composition may include a film former. For example, the film former may include a (meth)acrylate acid copolymer. For example, the (meth)acrylate acid copolymer may include acrylates/octylacrylamide copolymer. For example, acrylates/octylacrylamide copolymer may be present in an amount of about 1.0 wt. % to about 2.0 wt. %, based on the total weight of the composition.

In any of the preceding embodiments, the sunscreen composition may include an isoparaffin, an organosilicone, an ester of naphthalene dicarboxylic acid, an emulsifier, or combinations thereof. For example, the sunscreen composition may include isododecane, isohexadacane, disiloxane, dimethicone, cyclopentasiloxane, diethylhexyl 2,6-naphthalate, polyhydroxystearic acid, or combinations thereof.

In any of the preceding embodiments, the solvent may include butyloctyl salicylate, isopropyl myristate, diisobutyl adipate, butyl oleate, diethylhexyl maleate, diethylhexyl 2,6-naphthalate, and capric/caprylic triglyceride. For example, butyloctyl salicylate, isopropyl myristate, diisobutyl adipate, butyl oleate, diethylhexyl maleate, diethylhexyl 2,6-naphthalate, and capric/caprylic triglyceride may be present a ratio of 1:1:1:1:1:1:2.

In any of the preceding embodiments, the crystalline sunscreen mixture may include diethylhexyl butamido triazone, bis-ethylhexyloxyphenol methoxyphenyl triazine, butyl methoxydibenzoylmethane, and diethylamino hydroxybenzoyl hexyl benzoate. For example, diethylhexyl butamido triazone, bis-ethylhexyloxyphenol methoxyphenyl triazine, butyl methoxydibenzoylmethane, and diethylamino hydroxybenzoyl hexyl benzoate may be present in a ratio of 1:1.25:0.5:0.3 to 1:2:1.3:1.

In any of the preceding embodiments, the crystalline sunscreen mixture may include diethylhexyl butamido triazone and bis-ethylhexyloxyphenol methoxyphenyl triazine, and may exclude butyl methoxydibenzoylmethane and diethylamino hydroxybenzoyl hexyl benzoate. For example, the crystalline sunscreen mixture may include diethylhexyl butamido triazone, bis-ethylhexyloxyphenol methoxyphenyl triazine, butyl methoxydibenzoylmethane, and diethylamino hydroxybenzoyl hexyl benzoate in a ratio of 1:1.8:0:0.

In any of the preceding embodiments, the sunscreen composition may include a propellant. For example, the propellant may include isobutane, butane, propane, dimethyl ether, methyl ether, or combinations thereof.

In any of the preceding embodiments, the crystalline sunscreen mixture may include a sunscreen filter selected from a p-aminobenzoic acid, a benzophenone, a camphor, a cinnamate, a dibenzoylmethane, a salicylate, an imidazole, a triazole, a triazine, a triazole, triazone, a metal oxide, or combinations thereof.

In any of the preceding embodiments, the crystalline sunscreen mixture may exclude benzophenone-3, octyl methoxycinnamate, 2-ethylhexyl-2-cyano-3,3-diphenylacrylate, 3,3,5-trimethylcyclohexyl salicylate, and 2-ethylhexyl salicylate.

In any of the preceding embodiments, the sunscreen composition may further include one or more film formers, water, surfactants, emulsifiers, alcohols, rheology agents, skin conditioners, waxes, sterols, amino acids, colorants, dyes, pigments, powders, pH adjusters, fragrances, vitamins, preservatives, preservative boosters, foaming agents, chelating agents, cleansers, stabilizers, dispersants, essential fatty acids, sphingolipids, self-tanning compounds, fillers, antioxidants, minerals, keratolytics, retinoids, hormonal compounds, anti-aging agents, alpha-keto acids, anti-microbial agents, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin pollution protectants, skin penetration enhancers, exfoliants, lubricants, staining agents, depigmenting agents, hypopigmenting agents, extracts, elastomers, propellants, or combinations thereof.

Embodiments further relate to a method to protect a keratinous surface from UV radiation including contacting the keratinous surface with the sunscreen composition of any of the preceding embodiments. Embodiments further relate to a method to manufacture a sunscreen composition of any the preceding embodiments using traditional formulation processes, comprising at least mixing a solvent and one or more sunscreen filters to dissolve the sunscreen filters in the solvent. Embodiments further relate to a consumer packaged product including the sunscreen composition of any of the preceding embodiments.

### BRIEF DESCRIPTION OF THE DRAWINGS

The features of the disclosure believed to be novel and the elements characteristic of the invention are set forth with particularity in the appended claims. The figures are for illustration purposes only and are not drawn to scale. The disclosure itself, however, both as to organization and method of operation, can best be understood by reference to the description of the preferred embodiment(s) which follows, taken in conjunction with the accompanying drawings in which:

FIG. 1A to FIG. 1C are depictions illustrating solubility attributes for example sunscreen compositions including crystalline filters and solvent according to embodiments; and

FIG. 2 is a depiction illustrating migration attributes for example sunscreen compositions including crystalline filters and solvent according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %) based on a total weight of the composition unless otherwise indicated. Where appropriate, the International Nomenclature of Cosmetic Ingredients (INCI) name of ingredients/components is provided. In this regard, the term “(and)” is an INCI designation for a mixture of listed components separated by the term. Any numerical range recited herein is intended to include all sub-ranges subsumed therein, and such ranges are understood to include each and every number and/or fraction between the stated range lower and upper values. Moreover, the term “about” may refer to deviations of +20% unless otherwise stated.

### Sunscreen Filter(s)

A sunscreen composition includes a sunscreen filter capable of substantially absorbing, scattering, and/or deflecting UV radiation on a keratinous substrate. Preferably, the sun protection factor (SPF) of a sunscreen composition is ≥8 and the UVA protection factor (UVAPF) is at least ⅓ the SPF. Thus, for example, physical or mineral sunscreen filters include metal oxides (e.g., titanium dioxide, zinc oxide, combinations thereof, etc.) that may be used in effective amounts to provide an SPF of 8 or more (in contrast to other uses as colorants, etc.). Such filters may have various morphologies such as crystalline form (e.g., rutile, etc.), size (e.g., nano, non-nano, etc.), shape (e.g., amorphous, spherical, etc.), and functionalization (e.g., coatings, etc.) that allow for the effective use as sunscreen filters.

A sunscreen filter may include organic sunscreen filters such as, without limitation: p-aminobenzoic acids such as p-aminobenzoic acid (CAS #: 150-13-0), p-aminobenzoic acid, monoglyceryl ester (CAS #: 136-44-7), p-aminobenzoic acid, octyl dimethyl ester (padimate-0, CAS #: 21245-02-3), p-aminobenzoic acid, ethyl dihydroxypropyl (roxadimate, CAS #: 58882-17-0), p-aminobenzoic acid, 2,4-dihydroxy-N-(3-hydroxypropyl)-3,3-dimethylbutanamide ester, p-aminobenzoic acid, PEG-25 (CAS #: 116242-27-4); anthranilates such as menthyl anthranilate (CAS #: 134-09-8); benzophenones such as benzophenone (CAS #: 119-61-9), 2,4-dihydroxybenzophenone (benzophenone-1, CAS #: 131-56-6), 2,21,4,4′-tetrahydroxybenzophenone (benzophenone-2, CAS #: 131-55-5), 2-hydroxy-4-methoxybenzophenone (benzophenone-3, oxybenzone, CAS #: 131-57-7), 2-hydroxy-4-methoxybenzophenone-5-sulfonic acid (benzophenone-4, sulisobenzone, CAS #: 4065-45-6), 2-hydroxy-4-methoxybenzophenone-5-sulfonic acid, monosodium salt (benzophenone-5, CAS #: 6628-37-1), 2,2′-dihydroxy-4,4′-dimethoxybenzophenone (benzophenone-6, CAS #: 131-54-4), 5-chloro-2-hydroxybenzophenone (benzophenone-7, CAS #: 85-19-8), 2,2′-dihydroxy-4-methoxybenzophenone (benzophenone-8, dioxybenzone, CAS #: 131-53-3), 2,2′-dihydroxy-4,4′-dimethoxybenzophenone-5,5′-disulfonic acid sodium salt (benzophenone-9, CAS #: 76656-36-5), 2-hydroxy-4-methoxy-4′-methyl-benzophenone (benzophenone-10, Mexenone, CAS #: 1641-17-4), bis(2,4-ihydroxyphenyl)methanone (benzophenone-11, CAS #: 1341-54-4), 2-hydroxy-4-(octyloxy)benzophenone (benzophenone-12, octabenzone, CAS #: 1843 May 6), 2,2′-dihydroxy-4-methoxybenzophenone (dioxybenzone, CAS #: 131-53-3), 2-hydroxy-4-methoxybenzophenone (oxybenzone, CAS #: 131-57-7), 2-hydroxy-4-methoxybenzophenone-5-sulphonic acid (sulisobenzone, CAS #: 4065-45-6), hexyl 2-(4-(diethylamino)-2-hydroxybenzoyl)benzoate (CAS #: 302776-68-7); camphors such as benzylidene camphor sulfonic acid (CAS #: 56039-58-8), 3-benzylidene camphor (CAS #: 15087-24-8), polyacrylamidomethyl benzylidene camphor (CAS #: 113783-61-2), terephthalylidene dicamphor sulfonic acid (CAS #: 90457-82-2), 3-(4-methylbenzyliden)camphor (CAS #: 36861-47-9), camphor benzalkonium methosulfate (CAS #: 52793-97-2), bornelone (CAS #: 2226 Nov. 1); cinnamates such as ethyl cinnamate (CAS #: 103-36-6), 2-ethoxyethyl-p-methoxycinnamate (cinoxate, CAS #: 104-28-9), isoamyl p-methoxycinnamate (CAS #: 71617-10-2), diisopropyl methyl cinnamate (CAS #: 32580-71-5), 2-ethylhexyl alpha-cyano-beta-phenylcinnamate (octocrylene, CAS #: 6197-30-4), diethanolamine methoxycinnamate (CAS #: 56265-46-4), isopropyl methoxycinnamate (CAS #: 5466-76-2), isoamyl p-methoxycinnamate (CAS #: 71617-10-2), glyceryl octanoate dimethoxycinnamate, ethyl diisopropylcinnamate (CAS #: 32580-72-6), ethyl methoxycinnamate (CAS #: 99880-64-5), octyl methoxycinnamate (octinoxate, CAS #: 5466-77-3); dibenzoylmethanes such as dibenzoylmethane (CAS #: 120-46-7), isopropyl dibenzoylmethane (eusolex, CAS #: 63250-25-9), 4-tert-Butyl-4′-methoxy-dibenzoylmethane (avobenzone, CAS #: 70356-09-1); salicylates such as 3,3,5-trimethylcyclohexyl salicylate (homosalate, CAS #: 118-56-9), benzyl salicylate (CAS #: 118-58-1), octyl salicylate (octisalate, CAS #: 118-60-5), 2-hydroxyethyl salicylate (CAS #: 87-28-5), methyl salicylate (CAS #: 89-46-3), isopropylbenzyl salicylate (CAS #: 94134-93-7); imidazoles such as phenylbenzimidazole, phenylbenzimidazol-5-sulfonic acid (CAS #: 27503-81-7), phenylbenzimidazole tea sulfonate (CAS #: 73705-00-7), urocanic acid [4-imidazoleacrylic acid, CAS #: 104-98-3], ethyl urocanate (CAS #: 27538-35-8), bisymidazylate (CAS #: 180898-37-7), sodium phenylbenzimidazole sulfonate (CAS #: 5997-53-5); triazines such as bis(ethylhexyloxyphenol methoxyphenol) triazine (bemotrizinol, CAS #: 187393-00-6); triazoles such as 2-(2-hydroxy-5-methyl-phenyl)benzotriazole (drometrizole, CAS #: 2440-22-4); triazones such as diethylhexyl butamido triazone (iscotrizinol, CAS #: 154702-15-5), ethylhexyl triazone (CAS #: 88122-99-0); petrolatum jelly (CAS #: 8009-03-8), acrylates such as diurethane dimethacrylate (CAS #: 103597-45-1), ethyl 2-cyano-3,3-diphenylacrylate (etocrilene, CAS #: 5232-99-5); siloxanes such as drometrizole trisiloxane (CAS #: 155633-54-8); or combinations thereof.

A sunscreen filter that protects against UVA radiation may include avobenzone (e.g., Parasol® 1789), diethylamino hydroxybenzoyl hexyl benzoate (e.g., Uvinul® A Plus), terephthalylidene dicamphor sulfonic acid, bis-disulizole disodium, disodium phenyl dibenzimidazole tetrasulfonate, bis-diethylamino hydroxybenzoyl benzoate, bis-benzoxazolylphenyl ethylhexylamino triazine, or combinations thereof. A sunscreen filter that protects against UVB radiation may include octocrylene, octinoxate, octisalate, homosalate, ensulizole, ethylhexyl triazone (e.g., Uvinul® T150), enzacamene, amiloxate, iscotrizinol (e.g., Uvasorb® HEB), benzylidene malonate polysiloxane, padimate-O, trolamine salicylate, cinoxate, PABA, or combinations thereof. A sunscreen filter that protects against UVA and UVB radiation may include oxybenzone, meradimate, titanium dioxide, zinc oxide, bis-octrizole, bis-ethylhexyloxyphenol methoxyphenyl triazine (e.g., Tinosorb® S), drometrizole trisiloxane, sulisobenzone, dioxybenzone, or combinations thereof. A sunscreen filter that protects against damaging radiation within other regions in the electromagnetic spectrum, such as blue light (380 nm to 500 nm) or IR (700 nm-1 mm), may include, without limitation, metal oxides (e.g., titanium dioxide, zinc oxide, combinations thereof, etc.).

Accordingly, a sunscreen filter may be selected based on an ability to substantially absorb, scatter, and/or deflect UV radiation on a keratinous substrate. Additionally or alternatively, a sunscreen filter may be selected based on a solubility in an aqueous phase and/or a lipid phase. For example, the partition coefficient (P) may be used to determine the propensity of a neutral (uncharged) compound to dissolve in an immiscible biphasic system of lipid (fats, oils, organic solvents) and water. Thus, a sunscreen filter may be selected based on a determination of a negative value for log P indicating a higher affinity for the aqueous phase (more hydrophilic) or a positive value for log P indicating a higher affinity for the lipid phase (more lipophilic). Log P values for sunscreen filters are readily available.

A sunscreen filter may, for example, be present in an amount of about 1.0 wt. % to 40.0 wt. %, based on the total weight of the sunscreen composition. For example, one or more sunscreen filters may be present in an amount of 0.8 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 11.0 wt. %, 12.0 wt. %, 13.0 wt. %, 14.0 wt. %, 15.0 wt. %, 16.0 wt. %, 17.0 wt. %, 18.0 wt. %, 19.0 wt. %, 20.0 wt. %, 25.0 wt. %, 30.0 wt. %, 35.0 wt. %, 40.0 wt. %, 45.0 wt. %, 48.0 wt. %, including every number and/or fraction there between, based on the total weight of the sunscreen composition.

Preferably, a mixture of sunscreen filters includes a crystalline UVA filter and a crystalline UVB filter. Generally, crystalline filters are substances in the form of crystals or containing crystals that have carbon atoms and that exhibit an ability to absorb UV radiation (crystalline organic sunscreen filters). In embodiments, the ratio of crystalline UVA filter to crystalline UVB filter may be about 3:1 to about 1:5 to provide a synergistic enhancement to solubility. For example, the ratio by weight of the crystalline UVA filter to the crystalline UVB filter may be 3.6:0.8, 0.8:6, 3:1, 2.5:1, 2:1, 1.5:1. 1:1, 1:1.5, 1:2, 1:2.5, 1:3, 1:3.5, 1:4, 1:4.5, 1:5, 1:5.5, 1:6, including every number and/or fraction there between. Example concentrations and ratios described are for illustration and may be varied based on, for example, SPF and/or UVAPF requirements in the sunscreen composition.

In embodiments, the crystalline UVA filter may include butyl methoxydibenzoylmethane and exclude (e.g., is free of) diethylamino hydroxybenzoyl hexyl benzoate, and the crystalline UVB filter may include diethylhexyl butamido triazone. In one example, the ratio of butyl methoxydibenzoylmethane to diethylhexyl butamido triazone may be 1.25:1 to 1:1.25. Additionally or alternatively, butyl methoxydibenzoylmethane may be present in an amount of about 4 wt. % to about 5 wt. % and diethylhexyl butamido triazone may be present in an amount of about 4 wt. % to about 6 wt. %, based on the total weight of the sunscreen composition. Additionally or alternatively, diethylhexyl butamido triazone may be present in an amount ≥80%, preferably from 100% to 120%, of the amount of butyl methoxydibenzoylmethane present in the sunscreen composition.

Additionally, the crystalline UVB filter may optionally further include ethylhexyl triazone. In this regard, ethylhexyl triazone may be present in an amount less than an amount of diethylhexyl butamido triazone present in the sunscreen composition. Additionally or alternatively, diethylhexyl butamido triazone may be present in an amount ≥80% of the total amount of all crystalline UVB sunscreen filters present in the sunscreen composition.

In embodiments, the crystalline UVA filter may include diethylamino hydroxybenzoyl hexyl benzoate and the crystalline UVB filter may include diethylhexyl butamido triazone, ethylhexyl triazone, or combinations thereof. In one example, the ratio of diethylamino hydroxybenzoyl hexyl benzoate to diethylhexyl butamido triazone or to ethylhexyl triazone may be from 1:1 to 1:5. Additionally or alternatively, diethylamino hydroxybenzoyl hexyl benzoate may be present in an amount of about 1 wt. % to about 5 wt. %, based on the total weight of the composition, and diethylhexyl butamido triazone may be present in an amount of about 1 wt. % to about 6 wt. % or ethylhexyl triazone may be present in an amount of about 1 wt. % to about 5 wt. %, based on the total weight of the composition.

Additionally, the crystalline UVA filter may optionally further include butyl methoxydibenzoylmethane. Notably, the solubility of butyl methoxydibenzoylmethane may surprisingly be enhanced by diethylamino hydroxybenzoyl hexyl benzoate in the presence of a UVB crystalline filter. In one example, the ratio of butyl methoxydibenzoylmethane to diethylamino hydroxybenzoyl hexyl benzoate may be from 5:1 to 1:5. For example, the ratio of butyl methoxydibenzoylmethane to diethylamino hydroxybenzoyl hexyl benzoate may preferably be from 4:1 to 5:1, to enhance the solubility at least of butyl methoxydibenzoylmethane. Additionally or alternatively, diethylamino hydroxybenzoyl hexyl benzoate may be present in an amount greater than zero and ≤25%, preferably ≤20%, of the total amount of butyl methoxydibenzoylmethane in the sunscreen composition. Additionally or alternatively, diethylamino hydroxybenzoyl hexyl benzoate may be present in an amount of about 0.1 wt. % to about 1 wt. % and butyl methoxydibenzoylmethane may be present in an amount of about 4 wt. % to about 5 wt. %, based on the total weight of the sunscreen composition. Thus, for example, the ratio of butyl methoxydibenzoylmethane to diethylamino hydroxybenzoyl hexyl benzoate and to either diethylhexyl butamido triazone or to ethylhexyl triazone may be 1:0.2:1.

Optionally, a mixture of sunscreen filters may further include a crystalline UVA/UVB filter to increase at least the SPF of the sunscreen composition. Preferably, the UVA/UVB filter does not have a dampening effect on the synergistic enhancement to solubility at least of the crystalline UVA filter in the presence of the crystalline UVB filter. For example, the crystalline UVA/UVB filter may include bis-ethylhexyloxyphenol methoxyphenyl triazine, which surprisingly has no effect on the synergistic combination of crystalline UVA filter and crystalline UVB filter. Bis-ethylhexyloxyphenol methoxyphenyl triazine may be present, for example, in an amount up to about 3 wt. %, based on the total weight of the composition.

Optionally, the sunscreen composition may exclude a liquid UVB filter selected from 2-ethylhexyl alpha-cyano-beta-phenylcinnamate (octocrylene), 3,3,5-trimethylcyclohexyl salicylate (homosalate), and/or octyl salicylate (octisalate). It was expected that the addition of crystalline sunscreen filters in the absence of such strong filter solubilizers would exacerbate solubility challenges. Additionally or alternatively, the sunscreen composition may intentionally exclude UVB filters selected from 2-hydroxy-4-methoxybenzophenone (oxybenzone) and/or octyl methoxycinnamate (octinoxate). It was expected that removal of such strong UVB filters would exacerbate challenges related to SPF levels. However, sunscreen compositions according to embodiments surprisingly and unexpectedly maintain a homogeneous and stable dispersion of crystalline sunscreen filters at comparable or even at higher concentration levels while reliably providing a wide range of SPF levels (including relatively high SPF levels of ≥50) despite the absence of such UVB filters.

### Solvent(s)

A sunscreen composition includes a solvent to solubilize one or more sunscreen filters. Preferably, the solvent includes an ester selected from an ester of naphthalene dicarboxylic acid, an ester of salicylic acid, an ester of adipic acid, an ester of maleic acid, an ester of neopentanoic acid, an ester of C₈-C₁₈ fatty acids, or combinations thereof. Useful esters of naphthalene dicarboxylic acid include diethylhexyl 2,6-naphthalate. Useful salicylates include butyloctyl salicylate, C₁₂-C₁₅ alkyl salicylate, capryloyl salicylic acid, hexydodecyl salicylate, isocetyl salicylate, isodecyl salicylate, tridecyl salicylate, or combinations thereof. Useful adipates include diisobutyl adipate, dibutyl adipate, diisopropyl adipate, diethylhexyl adipate, or combinations thereof. Useful maleates include diethylhexyl maleate, dioctyl maleate, or combinations thereof. Useful neopentanoates include isostearyl neopentanoate, neopentyl glycol diheptanoate, or combinations thereof. Useful oleates include butyl oleate, decyl oleate, or combinations thereof. Useful esters of C₈-C₁₈ fatty acids include caprylic/capric triglyceride, isopropyl myristate, isopropyl palmitate, coco caprylate, octyl palmitate, or combinations thereof. Optionally, the solvent includes a sorbitol derivative such as dimethyl isosorbide. Notably, polyhydroxystearic acid may also be used as a solvent ingredient to solubilize the mixture of organic crystalline sunscreen filter.

A total amount of solvent to dissolve one or more crystalline sunscreen filters may, for example, be an amount of about 1.0 wt. % to about 50.0 wt. %, based on the total weight of the sunscreen composition. For example, one or more esters may be present in an amount of 0.8 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, 10.0 wt. %, 11.0 wt. %, 12.0 wt. %, 13.0 wt. %, 14.0 wt. %, 15.0 wt. %, 16.0 wt. %, 17.0 wt. %, 18.0 wt. %, 19.0 wt. %, 20.0 wt. %, 25.0 wt. %, 30.0 wt. %, 35.0 wt. %, 40.0 wt. %, 45.0 wt. %, 50.0 wt. %, 55.0 wt. %, 60.0 wt. %, including every number and/or fraction therebetween, based on the total weight of the sunscreen composition.

### Additional Ingredient(s)

A sunscreen composition may include additional ingredients to provide particular functions or particular product forms. Such ingredients are commercially available for use in cosmetic or pharmaceutical compositions. For example, sunscreen compositions may include any of the ingredients discussed in detail herein or the like for product forms such as, without limitation, a gel, a lotion, a cream, a spray, a mist, a stick, a mousse, a foam, an ointment, a milk, a base, a concentrate, a mist, etc. The amount of additional ingredients may be present from about 0.0001 wt. % to about 99.0 wt. %, based on the total weight of the sunscreen composition. One or more additional ingredients may, for example, be present in an amount of 0.01 wt. %, 1.0 wt. %, 2.0 wt. %, 5.0 wt. %, 10.0 wt. %, 20.0 wt. %, 30.0 wt. %, 40.0 wt. %, 50.0 wt. %, 60.0 wt. %, 70.0 wt. %, 80.0 wt. %, 91.8 wt. %, including every number and/or fraction therebetween, based on the total weight of the sunscreen composition. A total amount of all additional ingredients may be sufficient to bring the total amount of the sunscreen composition up to 100 wt. %. It is also understood that similar ingredients to those discussed in detail herein fall within the true scope and spirit of the present disclosure.

Examples of additional ingredients in embodiments may include, without limitation, film formers, water, surfactants, emulsifiers, alcohol, rheology agents (absorbents, thickening, gelling), skin conditioners (moisturizers, humectants, emollients), waxes (natural, synthetic), sterols, amino acids, colorants, dyes, pigments, powders, pH adjusters, fragrances, vitamins, preservatives, preservative boosters, foaming agents, chelating agents, cleansers, stabilizers, dispersants, essential fatty acids, sphingolipids, self-tanning compounds, fillers, antioxidants, minerals, keratolytics, retinoids, hormonal compounds, anti-aging agents, alpha-keto acids, anti-microbial agents (anti-bacterial, anti-fungal, anti-viral), analgesics, anti-allergenic, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin pollution protectants, skin penetration enhancers, exfoliants, lubricants, staining agents, depigmenting agents, hypopigmenting agents, extracts (fruit, flower, plant), elastomers, propellants, or combinations thereof.

Embodiments may, therefore, include film former polymers capable of providing a substantially homogenous and/or structured hydro-lipidic film on skin owing to its viscosity-building properties. Generally, a film former polymer may be synthesized from two or more different monomers to form copolymers and/or may from individual polymer chains that are connected by bridging molecules or crosslinking agents to form crosslinked copolymers known as cross polymers. For example, film former polymers may include, without limitation, monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. Useful film former polymers may include crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, acrylates/C₁₂-C₂₂ alkyl methacrylate copolymer, acrylates/octylacrylamide copolymer, methyl dihydroabietate, trimethylsiloxysilicate, C₂₄-C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, shellac, stearyl/octyldodecyl citrate crosspolymer, or combinations thereof.

Notably, certain cross polymers may primarily or exclusively provide gelling, rheology modifier, binder, absorbent, or hair fixative functionality owing to their structure and/or activation. For example, single molecules of carbomers have a molecular weight of 500,000+ Daltons which when crosslinked increases to over 1 billion Daltons to thicken at marked degrees. Accordingly, while such cross polymers may be used for rheology purposes, particularly useful polymers to form films may have an average molecular weight (MW) of about 1,500,000 Daltons (DA) or less. Preferably, a film former polymer may include a (meth)acrylate film former polymer such as acrylates/C₁₂-C₂₂ alkyl methacrylate copolymer (300,000 Da), acrylates/octylacrylamide copolymer (1,000,000 Da), or combinations thereof.

A film former polymer may be dispersed in a non-aqueous solvent such as an alcohol, an ether, a fatty acid, a silicone fluid, an ester, a hydrocarbon, a fluorinated hydrocarbon, or combinations thereof. In one example, a film former polymer may be dissolved in isododecane, isohexadacane, diisopropyl adipate, isoparrafins (e.g., C₁₃-C₁₆ isoparrafins), alcohol (e.g., C₂-C₁₀ alcohols, etc.), organosilicone compounds (e.g., disiloxane, dimethicone, cyclopentasiloxane, etc.), or combinations thereof. A film former polymer may be present in an aqueous solvent, for example dispersed in water. In one example, a (meth)acrylate film former polymer may be present in a percent solids content of about 20 wt. % to 100 wt. %, for example 30 wt. % to 40 wt. % when admixed, based on the total weight of the dispersion.

Generally, a film former polymer may be present in an amount of about 0.1 wt. % to about 5.0 wt. %, based on the total weight of the sunscreen composition. For example, one or more film former polymers may be present in an amount of 0.08 wt. %, 0.9 wt. %, 1.0 wt. %, 2.0 wt. %, 3.0 wt. %, 4.0 wt. %, 5.0 wt. %, 5.5 wt. %, 6.0 wt. %, including every number and/or fraction therebetween, based on the total weight of the sunscreen composition.

Notably, conventional use levels of certain film former polymers may be insufficient to minimize the migration of solvent and/or of crystalline sunscreen filters in certain cases. Meanwhile, increasing the concentration of film former polymers may be at the expense of solubility, sensory attributes (e.g., more stickiness), cost, or combinations thereof. Surprisingly, acrylates/octylacrylamide copolymer present in an amount of about 1.0 wt. % to about 2.0%, based on the total weight of an alcohol-based sunscreen composition, is sufficient to allow for minimized migration while maintaining relatively good solubility, safety, sensory attributes, and cost in substantially anhydrous sunscreen compositions. Moreover, (meth)acrylate film former polymer does not precipitate under test or ambient conditions. Of various film formers tested, VA/butyl maleate/isobornyl acrylate copolymer exhibited good performance. Similarly, acrylates/C₁₂-C₂₂ alkyl methacrylate copolymer also exhibited good performance.

Embodiments may include cosmetically acceptable carriers such as, without limitation, water, vegetable oils, ethers (e.g., dicapryl ether, dimethyl isosorbide, etc.), alcohols (e.g., ethanol, isopropanol, etc.), fatty alcohols (e.g., cetyl alcohol, stearyl alcohol, behenyl alcohol, etc.), hydrocarbon oils (e.g., mineral oil, petrolatum, isoeicosane polyisobutene, etc.), waxes (plant waxes, animal waxes, paraffin waxes, ozokerite, synthetic waxes, etc.), isoparaffins (e.g., isooctane, isododecane, isohexadacane, etc.), organosilicones (e.g., disiloxane, dimethicone, cyclopentasiloxane, etc.), or combinations thereof.

Carriers may be present in an amount from about 0.01 wt. % to about 99.0 wt. %, based on the total weight of the personal care composition. One or more carriers may, for example, be present in an amount of 0.008 wt. %, 0.1 wt. %, 0.5 wt. %, 1.0 wt. %, 2.0. wt. %, 3.0 wt. %, 4.0 wt. %, 5.0 wt. %, 6.0 wt. %, 7.0 wt. %, 8.0 wt. %, 9.0 wt. %, 10.0 wt. %, 11.0 wt. %, 12.0, 13.0 wt. %, 14.0 wt. %, 15.0 wt. %, 16.0 wt. %, 17.0 wt. %, 18.0 wt. %, 19.0 wt. %, 20.0 wt. %, 25.0 wt. %, 30.0 wt. %, 35.0 wt. %, 40.0 wt. %, 45.0 wt. %, 50.0 wt. %, 60.0 wt. %, 70.0 wt. %, 80.0 wt. %, 90.0 wt. %, 99.0 wt. %, including every number and/or fraction therebetween, based on the total weight of a personal care composition.

Accordingly, embodiments may include skin conditioners (e.g., moisturizers, emollients, humectants, etc.) such as, without limitation, certain esters (e.g., 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, butylene glycol, hexylene glycol, 2,3-butanediol, 1,2-alkane diols such propylene glycol, 1,2-butanediol, pentylene glycol, ethyl hexanediol, caprylyl glycol, decylene glycol, etc.), natural oils (e.g., nut oil, seed oil, fruit oil, etc.), fatty acids (e.g., lanolin, petrolatum, mineral oil, etc.), or combinations thereof. Notably, however, embodiments may intentionally exclude one or more skin conditioners. For example, sunscreen compositions may be free of mineral oil, any benzoates, and/or benzoates, e.g., selected from alkyl benzoates, ethyhexyl benzoate, phenethyl benzoate, propylene glycol dibenzoate, or combinations.

Embodiments may include water that is intentionally added in an amount of 15.0 wt. % or less to form a substantially anhydrous sunscreen composition. Moreover, one or more ingredients may have associated with it absorbed and/or adsorbed water that can be carried into the otherwise substantially anhydrous personal care composition. For economic, sourcing, and/or formulation reasons, it may be disadvantageous to process hygroscopic materials to make them completely free of water. Accordingly, a substantially anhydrous sunscreen composition may include 15.0 wt. % or less, preferably 5.0 wt. % or less (e.g., ≤1.0 wt. %), of water based on a total weight of the sunscreen composition. For example, a cream composition may include a surfactant, an emulsifier, and water in an amount of 15.0 wt. % or less, based on a total weight of the cream composition. A substantially anhydrous sunscreen composition may also include a C₂-C₄ alcohol present in an amount of about 30 wt. % to about 70 wt. %, based on the total weight of the composition. For example, a spray format may include ethanol (denatured) in an amount of about 30 wt. % to about 70 wt. %.

Non-aqueous carriers may be used with or without gelling agents for various applications. Rheology modifiers, discussed in further detail below, may be used as gelling agents in hydrous or anhydrous sunscreen compositions. In one example, an oil may be present in a substantially anhydrous sunscreen composition in an amount of 50.0 wt. % or greater (e.g., >90 wt. %), based on the total weight of the sunscreen composition. In a further example, an oil gel may be formulated using an oil and a gelling agent (e.g., hydrogenated butylene/ethylene/styrene copolymer, hydrogenated ethylene/propylene/styrene copolymer, etc.) present in an amount of about 0.1 wt. % to about 5.0 wt. %, based on the total weight of the sunscreen composition. Oily-alcoholic gels may also include natural or synthetic oil or wax. Meanwhile, alcoholic gels may include one or more lower alcohols or polyols, such as ethanol, propylene glycol or glycerin, and a rheology modifier, such as siliceous earth. Oils may also form oil-in-oil or oil-in-silicone or silicone-in-oil emulsions (e.g., castor oil in silicone oil, silicone oil in silicone oil, etc.) to deliver water-insoluble active ingredients (e.g., sunscreen actives, insect repellants, skin care actives, etc.) to a keratinous substrate.

Similarly, isoparaffins may be useful non-aqueous carriers. For example, isododecane or isohexadecane may be used. Also useful may be C₄-C₂₀ alkyl ethers of polypropylene glycols, C₁-C₂₀ carboxylic acid esters of polypropylene glycols, di-C₈-C₃₀ alkyl ethers, or combinations thereof. For example, PPG-14 butyl ether, PPG-15 stearyl ether, dioctyl ether, and/or dodecyl octyl ether may be used. Meanwhile, silicones including those that are non-volatile or volatile and those that are water soluble or water insoluble, or combinations thereof, may include organo-substituted polysiloxanes, which are either linear or cyclic polymers of monomeric silicone/oxygen monomers. Organopolysiloxanes may be selected from a wide variety of silicones spanning a broad range of volatilities and viscosities. For example, organopolysiloxanes may be selected from polyalkylsiloxanes, alkyl substituted dimethicones, dimethiconols, polyalkylaryl siloxanes, or combinations thereof. Thus, silicones may include dimethicone, cetyl dimethicone, cetyl dimethicone copolyol, cyclomethicone, cyclopentasiloxane, dimethicone copolyol, or combinations thereof. Volatile non-aqueous carriers may be preferred in some aspects of embodiments.

Embodiments may include surfactants such as anionic, cationic, nonionic, amphoteric, or combinations thereof. Examples of useful anionic surfactants include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. Examples of useful cationic surfactants include, without limitation, alkylamines, alkylimidazolines, quaternary ammonium compounds (QAC), or combinations thereof. Examples of useful nonionic surfactants include, without limitation, alkanolamides, amine oxides, esterified carboxylic acids, ethoxylated alcohols, poloxamers, or combinations thereof. Examples of useful amphoteric/zwitterionic surfactants include, without limitation, betaines, sultaines, cocamphoacetates, or combinations thereof.

Examples of emulsifiers, or surfactants that can stabilize emulsions, may include W/O emulsifiers, oil-in-water (O/W) emulsifiers, or combinations thereof. In this regard, water may be intentionally added at an amount greater than 15.0 wt. % such as, for example, 25.0 wt. %, 45.0 wt. %, 55.0 wt. %, 65.0 wt. %, 75.0 wt. %, including every number and/or fraction therebetween, based on the total weight of a substantially hydrous or aqueous sunscreen composition. A W/O emulsifier may have a hydrophilic-lipophilic balance (HLB) of <7, preferably from 4 to 6. Examples of W/O emulsifiers include certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), glycerin and/or fatty acid esters (e.g., glyceryl stearate, etc.), or combinations thereof.

Meanwhile, an O/W emulsifier may have a HLB≥7, preferably 7 to 10. Examples of O/W emulsifiers include certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB greater than 8, etc. Examples of natural emulsifiers include cetearyl alcohol (and) cetearyl glucoside, glyceryl stearate (and) cetearyl alcohol (and) sodium stearoyl lactylate, cetearyl wheat straw glycosides (and) cetearyl alcohol, sorbitan olivate, cetearyl olivate (and) sorbitan olivate, or combinations thereof.

It is, however, understood that other emulsion formats may similarly be used such as oil-in-oil or oil-in-silicone emulsions (e.g., castor oil in silicone oil, etc.) or water-in-silicone emulsions to deliver ingredients (e.g., sunscreen filters, antioxidants, etc.) to a keratinous substrate. Silicone emulsifiers are commercially available (e.g., Shin-Etsu, under the KSG, USG, and KF trade name series), which are compatible with silicones, organic oils, and esters. Silicone emulsifiers may include, for example, polyether-modified alkyl-branched silicone emulsifiers such as cetyl PEG/PPG-10/1 dimethicone and lauryl PEG-8 dimethicone, polyether/alkyl co-modified branched silicone emulsifiers such as lauryl PEG-9 polydimethylsiloxyethyl dimethicone, polyglycerin/alkyl co-modified branched silicone emulsifiers such as lauryl polyglyceryl-3 polydimethyldiloxyethyl dimethicone, polyglycerin-modified branched silicone emulsifiers such as polyglyceryl-3 disolixane dimethicone, polyether-modified silicone emulsifiers such as PEG/PPG-20/22 butyl ether dimethicone, PEG-10 dimethicone, PEG-11 methyl ether dimethicone, PEG-3 dimethicone, PEG-32 methyl ether dimethicone, emulsifying silicone elastomer gel agents such as dimethicone/polyglycerin-3 crosspolymer, PEG-10/lauryl dimethicone crosspolymer, PEG-15/lauryl dimethicone crosspolymer, PEG-15/lauryl polydimethylsiloxyethyl dimethicone crosspolymer, dimethicone/phenyl vinyl dimethicone crosspolymer, or combinations thereof. Thus, for example, personal care compositions may include W/O emulsifiers such as lauryl PEG-8 dimethicone, cetyl PEG/PPG-10/1 dimethicone, lauryl polyglyceryl-3 polydimethylsiloxyethyl dimethicone, or combinations thereof.

Surfactants may be present from about 0.1 wt. % to about 20.0 wt. %, based on the total weight of the personal care composition. One or more surfactants may, for example, be present in an amount of 0.008 wt. %, 0.1 wt. %, 0.5 wt. %, 1.0 wt. %, 2.0. wt. %, 3.0 wt. %, 4.0 wt. %, 5.0 wt. %, 6.0 wt. %, 7.0 wt. %, 8.0 wt. %, 9.0 wt. %, 10.0 wt. %, 11.0 wt. %, 12.0, 13.0 wt. %, 14.0 wt. %, 15.0 wt. %, 16.0 wt. %, 17.0 wt. %, 18.0 wt. %, 19.0 wt. %, 20.0 wt. %, 21.0 wt. %, 22.0 wt. %, including every number and/or fraction therebetween, based on the total weight of the personal care composition.

Embodiments may include a thickener to enhance the stability of emulsions, create gels, and/or provide texture. Examples of useful thickeners include, without limitation, plant-based thickeners (e.g., xanthan gum, konjac gum, guar gum, acacia gum, cellulose derivatives such as hydroxycellulose, hydroxyethyl cellulose, etc.), mineral thickeners (e.g., silica, bentonite, magnesium aluminum silicate, etc.), lipid thickeners (e.g., cetyl alcohol, stearyl alcohol, waxes such as carnauba wax, stearic acid, etc.), synthetic thickeners (e.g., carbomer, acrylates/C₁₀-C₃₀ alkyl acrylate crosspolymer, etc.), or combinations thereof. Embodiments may include a binder to hold ingredients together. Binders may include, for example, dry binders, wet binders, or combinations thereof. For example, dry binders may include zinc stearate, magnesium myristate, lithium stearate, or combinations thereof. Meanwhile, wet binders may include hydrocarbons, esters, silicones, or combinations thereof. The binder may include a sugar alcohol such as, without limitations, mannitol, sorbitol, xylitol, erythritol, isomalt, lactitol, maltitol, or combinations thereof. Embodiments may include an absorbent to absorb an ingredient. An absorbent may include natural materials, synthetic materials, or combinations thereof. Natural materials include, without limitation, bentonite, amylodextrin, Avena Sativa, alumina, or combinations thereof. Synthetic materials include, without limitation, starch octenylsuccinate, silica, or combinations thereof.

Rheology modifiers may be present from about 0.01 wt. % to about 40.0 wt. %, based on the total weight of the personal care composition. A plant-based, mineral, or synthetic thickener may, for example, be present in an amount of 0.05 wt. %, 0.1 wt. %, 0.5 wt. %, 1.0 wt. %, 2.0 wt. %, 3.0 wt. %, 3.5 wt. %, 4.0 wt. %, including every number and/or fraction therebetween, based on the total weight of the personal care composition. Meanwhile, wax thickeners used as a solidifying agent may be present in an amount up to about 30.0 wt. % such as, for example, 1.0 wt. %, 2.0 wt. %, 5.0 wt. %, 18.0 wt. %, 25.0 wt. %, including every number and/or fraction therebetween, based on the total weight of the composition.

Embodiments may include skin care agents such as antioxidants to counter free radicals that damage biomolecules. Antioxidants may be obtained, for example, from the extracts of flowers, fruits, vegetables, nuts, berries, plants, or combinations thereof. Antioxidants may include polyphenols and catechin derivatives such as catechin, epigallocatechin, epicatechin, epicatechin gallate, epigallocatechin gallate, gallocatechin gallate, gallocatechin, or combinations thereof. Such constituents are effective scavengers of reactive oxygen species and may also function indirectly as antioxidants through their effects on transcription factors and enzyme activities. Additionally or alternatively, antioxidants may include carotenoids (e.g., beta-carotene, lycopene, lutein, zeaxanthin), additional polyphenols (e.g., resveratrol), selenium, vitamins (A, B, C, E), grape seed extract, green tea extract, Coenzyme Q10, derivatives thereof, or combinations thereof.

Further non-limiting examples of skin care agents that provide skin benefits may include, for example, anti-acne ingredients, anti-aging ingredients, exfoliation ingredients, or combinations thereof. Anti-acne actives may include, without limitation, benzoyl peroxide, salicylic acid, retinoids (e.g., adapalene, tretinoin, etc.), azelaic acid, antibiotics (clindamycin, erythromycin, sulfacetamide, etc.), sulfur ingredients, or combinations thereof. Anti-aging ingredients may include retinol, bakuchiol, or combinations thereof. For example, a skin care composition may include bakuchiol to improve skin tone, improve skin elasticity, smooth fine lines, smooth wrinkles, or combinations thereof. Bakuchiol may preferably be present with an oil-based skin conditioning agent selected from hemp oil, bisabolol, adansonia digitata seed oil, or combinations thereof.

Exfoliation particles may include any material suitable to exfoliate the skin such as, without limitation, fruit seeds, fruit stones, nut shells, ground or fibrous plant material, polymers (e.g., natural, synthetic, etc.), mineral composites, or combinations thereof. Chemical exfoliants may additionally or alternatively be used. For example, allantoin, also known as aluminum dihydroxy allantoinate, may have keratolytic properties to assist in desquamation, the shedding of the outermost membrane or layer of tissue from the inside out. Additionally or alternatively, allantoin may soothe injured skin and promote wound healing, crease the water contents of cells, promote cell proliferation and supports overall remodeling, smooth skin, stimulate fibroblasts (cells responsible for producing collagen), boost extracellular matrix synthesis, or combinations thereof.

Meanwhile, odor neutralizers or absorbers may include perfumes, mineral powders, polymers (e.g., polymers in salt form), or combinations thereof. Perfumes may include, without limitation, naturally derived fragrances (extracts, essential oils, etc.), synthetically derived fragrances (small molecules), or combinations thereof. Mineral powder may include, without limitation, silica, silicate, bentonite, hectorite, smectite, kaolin, saponite, carbonate, or combinations. Polymers may include, without limitation, n-soya-n-ethyl morpholinium ethosulfate, cyclodextrins, or combinations thereof. Thus, in one example, metal oxide silicates (e.g., calcium silicate) may act as odor absorbents and neutralizers to absorb and neutralize body malodors. In a further example, cyclodextrins may be used to entrap active ingredients and thereby control the release of polyols, anti-microbials, zinc salts, polymers, bicarbonate salts, chelating agents, zeolites and activated carbon. Similarly, silicates, silicas and carbonates may absorb moisture which may indirectly reduce malodour formation by eliminating cutaneous flora that cause the odor.

Skin care agents may, for example, be present in an amount of about 0.0008 wt. % to about 20.0 wt. %, based on the total weight of a personal care composition. For example, one or more skin care active agents may be present in an amount of 0.0008 wt. %, 0.1 wt. %, 0.4 wt. %, 1.0 wt. %, 2.0 wt. %, 3.0 wt. %, 4.0 wt. %, 5.0 wt. %, 10.0 wt. %, 12.0 wt. %, 15.0 wt. %, 20.0 wt. %, 22.0 wt. %, including every number and/or fraction therebetween, based on a total weight of a personal care composition.

Embodiments may include elastomers. Elastomers may include natural or synthetic materials, including polymers, that substantially resume their original shape when a deforming force is removed. Silicone elastomers, for example, are a broad class of cross-linked dimethicone polymers that form three-dimensional polymeric structures. Interstitial pore volume, defined by the cross-link density of the silicones, can be swollen (filled) with a solvent or even a relatively small active material. Silicone elastomers may refer to silicone crosspolymers, dimethicone crosspolymers, dimethicone/vinyl dimethicone crosspolymers, Polysilicone-11, and so on. Thus, for example, the elastomer may be selected from dimethicone/dimethicone crosspolymers, dimethicone/vinyldimethicone crosspolymers, silicone crosspolymers, silicone, or combinations thereof.

Silicone elastomers are commercially available (e.g., Shin-Etsu, under the KSG, USG, and KF trade name series). Thus, for example, silicone elastomers may include dimethicone/phenyl vinyl dimethicone crosspolymer, dimethicone/polyglycerin-3 crosspolymer, lauryl polydimethylsiloxyethyl dimethicone/bis-vinyldimethicone crosspolymer, PEG-10/lauryl dimethicone crosspolymer, PEG-15/lauryl dimethicone crosspolymer, PEG-15/lauryl polydimethylsiloxyethyl dimethicone crosspolymer, dimethicone/phenyl vinyl dimethicone crosspolymer, diphenylsilocy phenyl thimethicone, polyglyceryl-3/lauryl poilydimethylsiloxyethyl dimethicone crosspolymer, vinyl dimethicone/lauryl dimethicone crosspolymer, or combinations thereof. Notably, as described above, one or more silicone elastomers may operatively function as an emulsifying silicone elastomer gel agent in various formulation formats. Silicone elastomers are available as loose powders, as swollen mixtures in cosmetic solvents, and as dispersions in water. The basic polymers can be further compounded into anhydrous and aqueous formula bases. Creams and lotions may, for example, contain a substantial amount of silicone elastomer (e.g., up to about 20 wt. %, up to about 10 wt. %, etc.).

Embodiments may include a propellant in aerosol formats. Propellants may include, for example, isobutane, butane, propane, dimethyl ether, methyl ether, tetraflouroethane, 1,1-difluoroethane, or combinations thereof. Propellants generally exists as equilibrium of vapor and liquid and can be either dissolved in or miscible with the composition. Ratios of the concentrate to propellant can be adjusted to account for cost, aesthetics, and product performance objectives. In one embodiment, the sunscreen composition concentrate and propellant are added to an aerosol container in a ratio of about 90:10 to about 60:40. The aerosol propellant may, for example, be present in an amount of about 10 wt. % to about 60 wt. %, based on a total weight of the sunscreen composition (e.g., final composition).

### Sunscreen Format(s)

A sunscreen composition may include one or more formulation formats such as, without limitation, an emulsion (O/W, W/O, O/O, silicone emulsions, multiple emulsion, etc.), a gel, a lotion, a cream, a spray, a mist, a stick, a mousse, a foam, an ointment, a milk, a base, a concentrate, a mist, or combinations thereof. Such formats may initially or finally be substantially aqueous or substantially anhydrous. For example, a substantially anhydrous base may be used in an aqueous emulsion to form a lotion or a cream. Similarly, ingredients and use levels to make such formats may be readily adjusted and mixed in a single phase or in multiple phases to provide a desired commercial sunscreen product. Thus, for example, water content, alcohol content, oil content, emulsifiers, foaming agents, gelling agents, etc., may be included, omitted, adjusted, or mixed in a single phase or in multiple phases to provide a commercial sunscreen product that accounts for cost, aesthetics, and/or product performance objectives.

Moreover, a sunscreen composition can be filled into any number of bottles, tottles, tubes, packets, etc., having a variety of closing structures, pumps, sprays (i.e., aerosols, bag on valve), amongst others, or combinations thereof. A sunscreen composition may, for example, be formulated as a concentrate with a propellant to provide a continuous aerosol spray commercial product to be applied via a spray nozzle. A bag-on-valve system may also be used to deliver the sunscreen composition using compressed gas such as air, nitrogen, or carbon dioxide. It is understood that similar product formats to those discussed in detail herein fall within the true scope and spirit of the present disclosure for any desired commercial product.

### Example(s)

All equipment was cleaned and sanitized before solubility testing. Ingredients were mixed in single phase or in multiple phases as appropriate according to traditional methods. Solubility testing included freeze thaw cycles over multiple twenty-four (24) hour periods. Notably, freeze-thaw testing is a reasonable and traditional generally accepted procedure for evaluating and determining solubility. More rigorous multi-week evaluations of the candidate oil phases and formulations were conducted using additional prolonged cooling methods. Solubility was determined on a pass or fail basis using visual inspection or microscopic analysis at room temperature and/or after freeze-thaw cycles or prolonged cooling.

In vitro data was obtained using a Labsphere Ultraviolet Transmittance Analyzer (Model UV-2000 available from the Solar Light Company, Philadelphia, Pennsylvania). Samples of the sunscreen compositions were transferred by an adjustable pipette and uniformly applied to molded HD6 plates (roughness 6 μm) by finger. After application, the coated plate was dried. The sample plate was placed inside the Labsphere Analyzer. The readings were recorded by the analyzer and the calculation of the SPF and UVAFP values were based on the following equation:

$$\text{?}$$

$$\text{?} \text{indicates text missing or illegible when filed}$$

with built-in software: UV-2000 application Version 1.1.0.0, wherein Ex is the Commission Internationale de l'Eclairage (CIE) erythemal spectral effectiveness, S) is designated as the solar spectral irradiance, and T_{λ} is the spectral transmittance of the sample as measured on the UV-2000. An average of multiple readings were recorded as the in-vitro SPF value.

Table 1 shows example sunscreen compositions having a synergistic combination of butyl methoxydibenzoylmethane and diethylhexyl butamido triazone. In embodiments, the example sunscreen compositions in Table 1 may be used in any formulation format. In one example, the example sunscreen compositions may be used in anhydrous spray formats.

TABLE 1

| Component (INCI) | Description | Approx. wt. % |
|---|---|---|
| Example Main Ingredients | | |
| butyl methoxydibenzoylmethane | Sunscreen UVA Filter | 3.0-5.0 |
| diethylhexyl butamido triazone | Sunscreen UVB Filter | 1.0-3.0 |
| butly octyl salicylate, | Solvent | 1.0-2.5 |
| diisopropyl adipate | | |
| Example Additional Ingredients | | |
| C₁₂-C₁₅ Alkyl Benzoate | Skin Conditioner | 3.0-7.0 |
| octocrylene, homosalate, | UVB Filter, | 4.0-13.0 |
| ethylhexyl salicylate | Solubilizer | |
| Ethanol | Carrier | Q.S. to 100 |

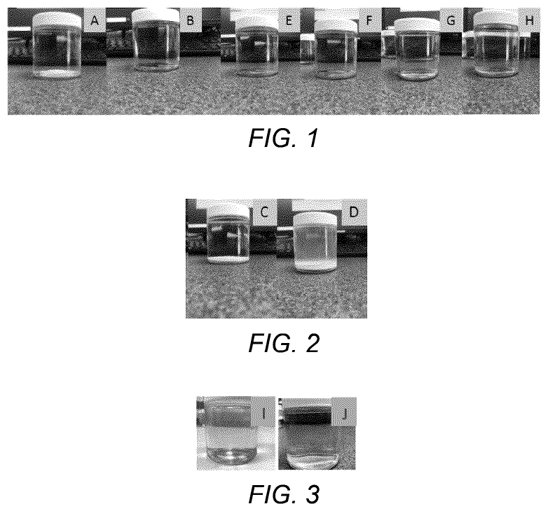

Referring to FIG. 1A, candidate formulations were made according to the levels listed in Table 1 having the same amounts of additional ingredients with the following levels of butyl methoxydibenzoylmethane and diethylhexyl butamido triazone, respectively: 5.0 wt. %, 0 wt. % (Sample A); 3.0 wt. %, 0 wt. % (Sample B); 3.0 wt. %, 1.0 wt. % (Sample E); 3.0 wt. %, 3.0 wt. % (Sample F); 3.0 wt. %, 5.0 wt. % (Sample G); 5.0 wt. %, 5.0 wt. % (Sample H). As shown in FIG. 1A, 5.0 wt. % of butyl methoxydibenzoylmethane in Sample A precipitates out of solution at room temperature. This amount of butyl methoxydibenzoylmethane can solubilize and does not precipitate out of solution after heating in an oven and at one freeze/thaw cycle. In contrast, 3.0 wt. % butyl methoxydibenzoylmethane in Sample B does not precipitate out of solution at room temperature. Meanwhile, adding or increasing diethylhexyl butamido triazone in Samples E-H unexpectedly does not cause precipitation even at higher levels of total crystalline sunscreen filter (Samples F-H) or at higher levels of butyl methoxydibenzoylmethane (Sample H) compared to Sample A. Moreover, no heat is needed to solubilize or maintain the mixture of crystalline sunscreen filters in solution.

Referring now to FIG. 1B, candidate formulations similar to Samples A-B and E-H were tested. Alcohol was removed for bis-ethylhexyloxyphenol methoxyphenyl triazine added to provide the following levels of butyl methoxydibenzoylmethane and bis-ethylhexyloxyphenol methoxyphenyl triazine, respectively: 3.0 wt. %, 1.0 wt. % (Sample C); 3.0 wt. %, 3.0 wt. % (Sample D). As shown in FIG. 1B, 1.0 wt. % bis-ethylhexyloxyphenol methoxyphenyl triazine in Sample C causes precipitation at room temperature even at the same use level in Sample B. No precipitation, however, is observed after heating in an oven. Moreover, 3.0 wt. % bis-ethylhexyloxyphenol methoxyphenyl triazine in Sample D causes precipitation out of solution after one freeze-thaw cycle. Thus, deleterious effects may exist from a mixture of butyl methoxydibenzoylmethane and bis-ethylhexyloxyphenol methoxyphenyl triazine.

As shown in FIG. 1C, candidate formulation Sample I similar to Sample G and candidate formulation J similar to Sample H were tested. In this regard, 5.0 wt. % alcohol replaced 2-ethylhexyl alpha-cyano-beta-phenylcinnamate removed. The mixture of crystalline filters in Sample I remained soluble at room temperature, while the mixture of crystalline filters in Sample J requires heat for solubilization. However, the SPF values and UVAPF values surprisingly increased in Samples I-J (122, 67) compared to Sample G-H (17, 20), respectively. Without being bound to any theory, increasing levels of diethylhexyl butamido triazone appear to surprisingly decrease SPF while the removal of 2-ethylhexyl alpha-cyano-beta-phenylcinnamate appears to surprisingly increase available solvent for use by diethylhexyl butamido triazone, improving solubility and increasing absorption efficacy.

Table 2 shows example sunscreen compositions having a synergistic combination of crystalline sunscreen filters. In addition, Table 2 shows example sunscreen compositions having a synergistic combination of crystalline sunscreen filters and solvent. In embodiments, the example sunscreen compositions in Table 2 may be used in any formulation format. In one example, the example sunscreen compositions may be used in aqueous formats. Thus, ingredients such as alcohol, water, emulsifiers, film formers, etc., may be readily included.

TABLE 2

| | Sample | | | | | | | | | | | | | | | |
|---|---|---|---|---|---|---|---|---|---|---|---|---|---|---|---|---|
| Ingredient(s) | K | L | M | N | O | P | Q | R | S | T | U | V | W | X | Y | Z |
| Butyl Methoxy- | 5 | 5 | 5 | 5 | 5 | 5 | 5 | 5 | 5 | 5 | 5 | 5 | 5 | 5 | 5 | 5 |
| dibenzoylmethane | | | | | | | | | | | | | | | | |
| Diethylamino | 0 | 0 | 0 | 0 | 0 | 0 | 0 | 0 | 0 | 0 | 0 | 0 | 0 | 0 | 0 | 0 |
| Hydroxybenzoyl Hexyl | | | | | | | | | | | | | | | | |
| Benzoate | | | | | | | | | | | | | | | | |
| Bis-ethylhexyloxyphenol | 3 | 3 | 3 | 3 | 3 | 3 | 3 | 3 | 3 | 3 | 3 | 3 | 3 | 3 | 3 | 3 |
| Methoxyphenyl Triazine | | | | | | | | | | | | | | | | |
| Diethylhexyl Butamido | 5 | 5 | 5 | 5 | 5 | 5 | 5 | 5 | 5 | 5 | 5 | 5 | 5 | 5 | 5 | 5 |
| Triazone | | | | | | | | | | | | | | | | |
| Ethylhexyl Triazone | 0 | 0 | 0 | 0 | 0 | 0 | 0 | 0 | 0 | 0 | 0 | 0 | 0 | 0 | 0 | 0 |
| Butyl Octyl Salicylate | 5 | 5 | 5 | 5 | 5 | 5 | 5 | 5 | 5 | 5 | 5 | 5 | 5 | 5 | 5 | 5 |
| Isopropyl Myristate | 10 | 8 | 12.5 | 0 | 0 | 5 | 5 | 10 | 7.5 | 0 | 0 | 0 | 0 | 0 | 10 | 0 |
| Diisobutyl Adipate | 10 | 0 | 0 | 20 | 0 | 0 | 5 | 0 | 0 | 0 | 0 | 0 | 0 | 0 | 0 | 0 |
| Dibutyl Adipate | 10 | 8 | 12.5 | 0 | 20 | 5 | 0 | 10 | 0 | 10 | 10 | 0 | 0 | 0 | 10 | 0 |
| Caprylic/Capric TG | 0 | 0 | 0 | 0 | 0 | 0 | 0 | 0 | 0 | 10 | 10 | 10 | 10 | 10 | 0 | 10 |
| Diethylhexyl Maleate | 0 | 0 | 0 | 0 | 0 | 5 | 5 | 5 | 13 | 0 | 0 | 0 | 0 | 0 | 0 | 0 |
| Butyl Oleate | 0 | 0 | 0 | 0 | | 0 | 0 | 0 | 10 | 0 | 0 | 10 | 0 | 0 | 0 | 0 |
| Dimethyl Isosorbide | 0 | 0 | 0 | 0 | | 0 | 0 | 0 | 0 | 0 | 0 | 0 | 10 | 0 | 0 | 0 |
| Diethylhexyl adipate | 0 | 0 | 0 | 0 | 0 | 0 | 0 | 0 | 0 | 0 | 0 | 0 | 0 | 10 | 0 | 0 |
| Isostearyl | 0 | 0 | 0 | 0 | 0 | 0 | 0 | 0 | 0 | 0 | 0 | 0 | 0 | 0 | 0 | 10 |
| Neopentanoate | | | | | | | | | | | | | | | | |
| Decyl Oleate | 0 | 0 | 0 | 0 | 0 | 0 | 0 | 0 | 0 | 0 | 0 | 0 | 0 | 0 | 0 | 0 |
| Diisopropyl Adipate | 0 | 0 | 0 | 0 | 0 | 0 | 0 | 0 | 0 | 0 | 0 | 0 | 0 | 0 | 0 | 0 |
| Coco Caprylate | 0 | 0 | 0 | 0 | 0 | 0 | 0 | 0 | 0 | 0 | 0 | 0 | 0 | 0 | 0 | 0 |
| Total wt. % | 48 | 33 | 43 | 38 | 38 | 33 | 33 | 43 | 38 | 38 | 38 | 38 | 38 | 38 | 38 | 38 |
| (Formulation) | | | | | | | | | | | | | | | | |
| Solubility (Pass, Fail) | P | F | P | P | F | P | P | P | P | P | P | P | P | P | F | P |

| | Sample | | | | | | | | | | | | | | | |
|---|---|---|---|---|---|---|---|---|---|---|---|---|---|---|---|---|
| Ingredient(s) | A1 | B1 | C1 | D1 | E1 | F1 | G1 | H1 | I1 | J1 | K1 | L1 | M1 | N1 | O1 | P1 |
| Butyl Methoxy- | 5 | 5 | 5 | 5 | 5 | 5 | 5 | 5 | 5 | 5 | 5 | 5 | 5 | 5 | 5 | 5 |
| dibenzoylmethane | | | | | | | | | | | | | | | | |
| Diethylamino | 0 | 0 | 0 | 0 | 0 | 0 | 0 | 0 | 0 | 0 | 0 | 0 | 0 | 0 | 0 | 0 |
| Hydroxybenzoyl Hexyl | | | | | | | | | | | | | | | | |
| Benzoate | | | | | | | | | | | | | | | | |
| Bis-ethylhexyloxyphenol | 3 | 3 | 3 | 3 | 3 | 0 | 3 | 3 | 3 | 3 | 3 | 3 | 3 | 3 | 3 | 3 |
| Methoxyphenyl Triazine | | | | | | | | | | | | | | | | |
| Diethylhexyl Butamido | 5 | 5 | 5 | 5 | 6 | 5 | 0 | 5 | 5 | 5 | 5 | 5 | 5 | 5 | 5 | 5 |
| Triazone | | | | | | | | | | | | | | | | |
| Ethylhexyl Triazone | 0 | 0 | 0 | 0 | 0 | 0 | 0 | 0 | 0 | 0 | 0 | 0 | 0 | 0 | 0 | 0 |
| Butyl Octyl Salicylate | 5 | 5 | 5 | 8 | 5 | 5 | 5 | 5 | 5 | 5 | 5 | 5 | 5 | 5 | 5 | 5 |
| Isopropyl Myristate | 0 | 0 | 0 | 0 | 0 | 0 | 0 | 0 | 0 | 0 | 0 | 0 | 0 | 0 | 0 | 0 |
| Diisobutyl Adipate | 0 | 0 | 5 | 4 | 5 | 5 | 5 | 0 | 0 | 0 | 0 | 0 | 0 | 0 | 0 | 0 |
| Dibutyl Adipate | 0 | 0 | 5 | 4 | 5 | 5 | 5 | 5 | 10 | 10 | 10 | 10 | 10 | 10 | 10 | 8 |
| Caprylic/Capric TG | 10 | 10 | 10 | 9 | 10 | 10 | 10 | 10 | 0 | 0 | 0 | 0 | 0 | 0 | 0 | 0 |
| Diethylhexyl Maleate | 0 | 0 | 0 | 0 | 0 | 0 | 0 | 0 | 0 | 0 | 0 | 0 | 0 | 0 | 10 | 8 |
| Butyl Oleate | 0 | 0 | 0 | 0 | 0 | 0 | 0 | 0 | 10 | 0 | 0 | 0 | 0 | 0 | 0 | 0 |
| Dimethyl Isosorbide | 0 | 0 | 0 | 0 | 0 | 0 | 0 | 0 | 0 | 10 | 0 | 0 | | 0 | 0 | 0 |
| Diethylhexyl adipate | 0 | 0 | 0 | 0 | 0 | 0 | 0 | 0 | 0 | 0 | 10 | 0 | 0 | 0 | 0 | 0 |
| Isostearyl | 0 | 0 | 0 | 0 | 0 | 0 | 0 | 0 | 0 | 0 | 0 | 10 | 0 | 0 | 0 | 0 |
| Neopentanoate | | | | | | | | | | | | | | | | |
| Decyl Oleate | 10 | 0 | 0 | 0 | 0 | 0 | 0 | 0 | 0 | 0 | 0 | 0 | 10 | 0 | | 0 |
| Diisopropyl Adipate | 0 | 0 | 0 | 0 | 0 | 0 | 0 | 5 | 0 | 0 | 0 | 0 | 0 | 0 | 0 | 0 |
| Coco Caprylate | 0 | 10 | 0 | 0 | 0 | 0 | 0 | 0 | 0 | 0 | 0 | 0 | 0 | 10 | 0 | 0 |
| Total wt. % | 38 | 38 | 38 | 38 | 39 | 35 | 33 | 38 | 38 | 38 | 38 | 38 | 38 | 38 | 38 | 34 |
| (Formulation) | | | | | | | | | | | | | | | | |
| Solubility (Pass, Fail) | P | F | P | P | P | P | F | P | P | P | P | P | P | P | P | F |

Sample

| Ingredient(s) | Q1 | R1 | S1 | T1 | U1 | V1 | W1 | X1 | Y1 | Z1 | A2 | B2 | C2 | D2 | E2 | F2 |
|---|---|---|---|---|---|---|---|---|---|---|---|---|---|---|---|---|
| Butyl Methoxy- | 5 | 5 | 5 | 5 | 5 | 5 | 5 | 5 | 5 | 5 | 5 | 5 | 5 | 4 | 5 | 5 |
| dibenzoylmethane | | | | | | | | | | | | | | | | |
| Diethylamino | 0 | 0 | 0 | 0 | 0 | 0 | 0 | 0 | 0 | 0 | 0 | 0 | 0 | 0 | 0 | 1 |
| Hydroxybenzoyl Hexyl | | | | | | | | | | | | | | | | |
| Benzoate | | | | | | | | | | | | | | | | |
| Bis-ethylhexyloxyphenol | 3 | 3 | 3 | 3 | 3 | 3 | 3 | 3 | 3 | 3 | 3 | 3 | 3 | 5 | 3 | 3 |
| Methoxyphenyl Triazine | | | | | | | | | | | | | | | | |
| Diethylhexyl Butamido | 5 | 5 | 5 | 5 | 5 | 5 | 5 | 5 | 5 | 5 | 5 | 5 | 5 | 5 | 0 | 5 |
| Triazone | | | | | | | | | | | | | | | | |
| Ethylhexyl Triazone | 0 | 0 | 0 | 0 | 0 | 0 | 0 | 0 | 0 | 0 | 0 | 0 | 0 | 0 | 5 | 0 |
| Butyl Octyl Salicylate | 8 | 8 | 8 | 5 | 9 | 8 | 7 | 6 | 5 | 0 | 8 | 5 | 5 | 0 | 5 | 5 |
| Isopropyl Myristate | 0 | 0 | 0 | 0 | 0 | 0 | 0 | 0 | 0 | 0 | 0 | 0 | 0 | 0 | 0 | 0 |
| Diisobutyl Adipate | 0 | 0 | 0 | 5 | 0 | 0 | 0 | 0 | 0 | 0 | 0 | 2 | 2 | 2 | 2 | 2 |
| Dibutyl Adipate | 9 | 7 | 6 | 5 | 7 | 6 | 5 | 4 | 7 | 8 | 6 | 8 | 8 | 8 | 8 | 8 |
| Caprylic/Capric TG | 0 | 0 | 6 | 5 | 7 | 6 | 5 | 4 | 7 | 8 | 6 | 10 | 10 | 10 | 10 | 10 |
| Diethylhexyl Maleate | 9 | 10 | 6 | 5 | 7 | 6 | 5 | 4 | 7 | 8 | 6 | 0 | 0 | 0 | 0 | 0 |
| Butyl Oleate | 0 | 0 | 0 | 0 | 0 | 0 | 0 | 0 | 0 | 0 | 0 | 0 | 0 | 0 | 0 | 0 |
| Dimethyl Isosorbide | 0 | 0 | 0 | 0 | 0 | 0 | 0 | 0 | 0 | 0 | 0 | 0 | 0 | 0 | 0 | 0 |
| Diethylhexyl adipate | 0 | 0 | 0 | 0 | 0 | 0 | 0 | 0 | 0 | 0 | 0 | 0 | 0 | 0 | 0 | 0 |
| Isostearyl | 0 | 0 | 0 | 0 | 0 | 0 | 0 | 0 | 0 | 0 | 0 | 0 | 0 | 0 | 0 | 0 |
| Neopentanoate | | | | | | | | | | | | | | | | |
| Decyl Oleate | 0 | 0 | 0 | 0 | 0 | 0 | 0 | 0 | 0 | 0 | 0 | 0 | 0 | 0 | 0 | 0 |
| Diisopropyl Adipate | 0 | 0 | 0 | 0 | 0 | 0 | 0 | 0 | 0 | 0 | 0 | 0 | 0 | 0 | 0 | 0 |
| Coco Caprylate | 0 | 0 | 0 | 0 | 0 | 0 | 0 | 0 | 0 | 0 | 0 | 0 | 0 | 0 | 0 | 0 |
| Total wt. % | 38 | 38 | 38 | 38 | 42 | 38 | 34 | 30 | 38 | 36 | 38 | 38 | 38 | 34 | 38 | 39 |
| (Formulation) | | | | | | | | | | | | | | | | |
| Solubility (Pass, Fail) | P | P | P | F | P | F | F | F | P | P | P | P | P | P | F | P |

| | Sample | | | | | | |
|---|---|---|---|---|---|---|---|
| Ingredient(s) | G2 | H2 | I2 | J2 | K2 | L2 | M2 |
| Butyl Methoxy- | 0 | 0 | 0 | 5 | 5 | 5 | 5 |
| dibenzoylmethane | | | | | | | |
| Diethylamino | 5 | 5 | 5 | 1 | 0 | 0 | 0 |
| Hydroxybenzoyl Hexyl | | | | | | | |
| Benzoate | | | | | | | |
| Bis-ethylhexyloxyphenol | 3 | 0 | 3 | 3 | 3 | 3 | 3 |
| Methoxyphenyl Triazine | | | | | | | |
| Diethylhexyl Butamido | 5 | 0 | 0 | 0 | 1 | 3 | 4 |
| Triazone | | | | | | | |
| Ethylhexyl Triazone | 0 | 5 | 5 | 5 | 4 | 3 | 1 |
| Butyl Octyl Salicylate | 5 | 5 | 5 | 5 | 5 | 5 | 5 |
| Isopropyl Myristate | 0 | 0 | 0 | 0 | 0 | 0 | 0 |
| Diisobutyl Adipate | 2 | 2 | 2 | 2 | 2 | 2 | 2 |
| Dibutyl Adipate | 8 | 8 | 8 | 8 | 8 | 8 | 8 |
| Caprylic/Capric TG | 10 | 10 | 10 | 10 | 10 | 10 | 10 |
| Diethylhexyl Maleate | 0 | 0 | 0 | 0 | 0 | 0 | 0 |
| Butyl Oleate | 0 | 0 | 0 | 0 | 0 | 0 | 0 |
| Dimethyl Isosorbide | 0 | 0 | 0 | 0 | 0 | 0 | 0 |
| Diethylhexyl adipate | 0 | 0 | 0 | 0 | 0 | 0 | 0 |
| Isostearyl | 0 | 0 | 0 | 0 | 0 | 0 | 0 |
| Neopentanoate | | | | | | | |
| Decyl Oleate | 0 | 0 | 0 | 0 | 0 | 0 | 0 |
| Diisopropyl Adipate | 0 | 0 | 0 | 0 | 0 | 0 | 0 |
| Coco Caprylate | 0 | 0 | 0 | 0 | 0 | 0 | 0 |
| Total wt. % | 38 | 35 | 38 | 39 | 38 | 38 | 38 |
| (Formulation) | | | | | | | |
| Solubility (Pass, Fail) | P | P | P | P | F | F | P |

Table 2 shows various example formulations having different combinations of crystalline sunscreen filters and solvent ingredients. In embodiments, a mixture of butyl methoxydibenzoylmethane and diethylhexyl butamido triazone synergistically maximizes the solubility (minimizes or prevents precipitation) of the mixture and specifically of butyl methoxydibenzoylmethane (e.g., Samples C1, G1). In one example, the ratio of butyl methoxydibenzoylmethane to diethylhexyl butamido triazone may be 1.25:1 to 1:1.25. For example, butyl methoxydibenzoylmethane may be present in an amount of about 4 wt. % to about 5 wt. % and diethylhexyl butamido triazone may be present in an amount of about 4 wt. % to about 6 wt. %, based on the total weight of the sunscreen composition. Moreover, the mixture may optionally include (e.g., Sample F2) or intentionally exclude (e.g., Sample M2) diethylamino hydroxybenzoyl hexyl benzoate.

In embodiments, diethylhexyl butamido triazone may be present in an amount ≥80% of the amount of butyl methoxydibenzoylmethane present in the sunscreen composition (e.g., Samples E1, D2, M2). Additionally or alternatively, diethylhexyl butamido triazone may be present in an amount ≥80% of the total amount of all crystalline UVA sunscreen filters present in the sunscreen composition (e.g., Sample F2). Additionally or alternatively, diethylhexyl butamido triazone may be present in an amount ≥80% of the total amount of all crystalline UVB sunscreen actives present in the sunscreen composition (e.g., Samples J2-M2). Moreover, the mixture of sunscreen filters may optionally include ethylhexyl triazone (e.g., Samples E2, H2-M2). Additionally, ethylhexyl triazone may be present in an amount less than the amount of diethylhexyl butamido triazone (e.g., Samples K2-M2).

In embodiments, a mixture of diethylamino hydroxybenzoyl hexyl benzoate and diethylhexyl butamido triazone or ethylhexyl triazone may also synergistically maximize the solubility and minimize the precipitation of the mixture (e.g., Samples F2-J2). Thus, for example, the ratio of diethylamino hydroxybenzoyl hexyl benzoate to diethylhexyl butamido triazone or to ethylhexyl triazone may be from 2:1 to 1:5. In one example, diethylamino hydroxybenzoyl hexyl benzoate may be present in an amount of about 1 wt. % to about 5 wt. % and diethylhexyl butamido triazone may be present in an amount of about 1 wt. % to about 10 wt. % or ethylhexyl triazone may be present in an amount of about 1 wt. % to about 5 wt. %, based on the total weight of the sunscreen composition. Optionally, the mixture of crystalline filters may include butyl methoxydibenzoylmethane with or without diethylhexyl butamido triazone (e.g., Samples F2-J2). Thus, the mixture may also synergistically maximize the solubility and minimize the precipitation of butyl methoxydibenzoylmethane.

In embodiments, a mixture of crystalline sunscreen filters may further optionally include a crystalline UVA/UVB filter. For example, the mixture may include bis-ethylhexyloxyphenol methoxyphenyl triazine. In one example, bis-ethylhexyloxyphenol methoxyphenyl triazine is present in an amount of about 3 wt. %, based on the total weight of the sunscreen composition. Notably, bis-ethylhexyloxyphenol methoxyphenyl triazine may provide additional photoprotection without impact to a synergistic combination of crystalline UVA filter and crystalline UVB filter in a sunscreen composition (e.g., Samples C₁, F1, H1).

Accordingly, butyl methoxydibenzoylmethane may be present in an amount of 0 wt. % to 5.0 wt. %, preferably 3.0 wt. % to 5.0 wt. % when present, diethylhexyl butamido triazone may be present in an amount of 0 wt. % to 6.0 wt. %, preferably 1.0 wt. % to 6.0 wt. % when present, diethylamino hydroxybenzoyl hexyl benzoate may be present in an amount of 0 wt. % to 5.0 wt. %, preferably 1.0 wt. % to 6.0 wt. % when present, bis-ethylhexyloxyphenol methoxyphenyl triazine may be present in an amount of 0 wt. % to 4.0 wt. %, preferably about 3.0 wt. % when present, and ethylhexyl triazone may be present in an amount of 0 wt. % to 5.0 wt. %, preferably 1.0 wt. 5.0 wt. % when present, based on the total weight of the sunscreen composition. Additionally or alternatively, the sunscreen composition may exclude 2-ethylhexyl alpha-cyano-beta-phenylcinnamate (octocrylene), 3,3,5-trimethylcyclohexyl salicylate (homosalate), octyl salicylate (octisalate), or combinations thereof.

Additionally, diethylhexyl butamido triazone may act synergistically with solvent to keep butyl methoxydibenzoylmethane and/or bis-ethylhexyloxyphenol methoxyphenyl triazine in solution. In embodiments, ethylhexyl triazone may be replaced with diethylhexyl butamido triazone such as when 1.0 wt. % to 5.0 wt. % diethylamino hydroxybenzoyl hexyl benzoate is present. When substituting ethylhexyl triazone with diethylhexyl butamido triazone, crystals may begin to form after ethylhexyl triazone exceeds 20 wt. % of the combination of the two at 5.0% total of the filters. In embodiments, bis-ethylhexyloxyphenol methoxyphenyl triazine may be present in an amount of 0 wt. % to 4.0 wt. %, preferably about 3.0 when present, based on the total weight of the sunscreen composition. In embodiments, 5.0 wt. % butyl methoxydibenzoylmethane and 3.0% bis-ethylhexyloxyphenol methoxyphenyl triazine may require more than 0 wt. % diethylhexyl butamido triazone.

Meanwhile, Table 2 further shows that embodiments may include a solvent composed of an ester of salicylic acid, an ester of adipic acid, an ester of maleic acid, an ester of neopentanoic acid, an ester of C₈₋₁₈ fatty acids, or combinations thereof. For example, the solvent may include butyloctyl salicylate, diisobutyl adipate, dibutyl adipate, diisopropyl adipate, diethylhexyl adipate, diethylhexyl maleate, isostearyl neopentanoate, butyl oleate, decyl oleate, caprylic/capric triglyceride, isopropyl myristate, coco caprylate, octyl palmitate, or combinations thereof. The solvent may include dimethyl isosorbide.

In embodiments, butyl octyl salicylate may be present in an amount of 0 wt. % to 9 wt. %, preferably 4 wt. % to 9 wt. % when present, caprylic/capric triglyceride may be present in an amount of 0 wt. % to 10 wt. %, preferably 5 wt. % to 10 wt. % when present, dibutyl adipate may be present in an amount of 0 wt. % to 13 wt. %, preferably 4 wt. % to 13 wt. % when present, and diisobutyl adipate may be present in an amount of 0 wt. % to 20 wt. %, preferably 2 wt. % to 20 wt. % when present, based on the total weight of the sunscreen composition. For example, the solvent may include butyloctyl salicylate, dibutyl adipate and/or diisobutyl adipate, and caprylic/capric triglyceride. In one example, a ratio of butyloctyl salicylate, dibutyl adipate and/or diisobutyl adipate, and caprylic/capric triglyceride may be 1:2:2.

Notably, solvent having butyloctyl salicylate, dibutyl adipate and/or diisopropyl adipate, and capric/caprylic triglyceride surprisingly takes 25 grams (g) to solubilize 13.5 g of butyl methoxydibenzoylmethane, diethylhexyl butamido triazone, diethylamino hydroxybenzoyl hexyl benzoate, bis-ethylhexyloxyphenol methoxyphenyl triazine, and ethylhexyl triazone. This is unexpected since it theoretically takes at least 37 g of the solvent combination to solvate 3 g of bis-ethylhexyloxyphenol methoxyphenyl triazine (a filter that is harder to solubilize than even avobenzone). Similarly, the theoretical amount of octocrylene, homosalate, octisalate, octinoxate, ethylhexyl benzoate and C₁₂-C₁₅ alkyl benzoate to solvate 3 g of bis-ethylhexyloxyphenol methoxyphenyl triazine is 21 g. Thus, solubilizers such as octocrylene, homosalate, octisalate, octinoxate, ethylhexyl benzoate, phenethyl benzoate, and/or C₁₂-C₁₅ alkyl benzoate may intentionally be omitted to address concerns while surprisingly solubilizing relatively more crystalline sunscreen filters with relatively less solvent content. Additional benefits from relatively less solvent content, such as migration benefits, are discussed below.

In embodiments, adipates equaling about 10 wt. % and caprylic/capric triglycerides present in an amount of about 10 wt. %, and/or a 1:1 ratio of adipates to triglycerides, with butyl octyl salicylate in an amount of 0 wt. % to 5 wt. % may solubilize a mixture of crystalline filters (e.g., Samples T, U, C1-F1, H1,B2-D2, F2-J2, M2). In embodiments, caprylic/capric triglyceride may also be replaced by butyl oleate, dimethyl isosorbide, diethylhexyl adipate, isostearyl neopentanoate, decyl oleate, diethylhexyl maleate, or coco caprylate. For example, 10 wt. % of caprylic/capric triglyceride may be replaced with one or more of the following (e.g., with 10 wt. % dibutyl adipate and 5 wt. % butyl octyl salicylate): butyl oleate (e.g., Sample V); dimethyl isosorbide (e.g., Sample W); diethylhexyl adipate (e.g., Sample X); isostearyl neopentanoate (e.g., Sample Z); decyl oleate (e.g., Sample A1); coco caprylate (e.g., Sample N1); or diethyhexyl maleate (e.g., 01). In this regard, diethylhexyl maleate and dibutyl adipate may be present at or above 8.5 wt. % each in the absence of caprylic/capric triglyceride. Moreover, half of caprylic/capric triglyceride may be replaced by diethylhexyl maleate (e.g., Sample T1). However, replacement by dibutyl adipate for a total level of 20 wt. % dibutyl adipate may impact solubility in certain embodiments (e.g., Sample O). Similarly, there may be an upper limit of 15 wt. % caprylic/capric triglyceride in the presence of at least 5.0 wt. % dibutyl adipate (not shown).

In embodiments, dibutyl adipate may be replaced with butyl oleate, dimethyl isosorbide, diethylhexyl adipate, isostearyl neopentonate, or decyl oleate. For example, 10 wt. % of dibutyl adipate may be replaced with one or more of the following (e.g., with 10 wt. % caprylic/capric triglyceride and 5 wt. % butyl octyl salicylate): butyl oleate (e.g., Sample U); dimethyl isosorbide (e.g., Sample W); diethylhexyl adipate (e.g., Sample X); isostearyl neopentanoate (e.g., Sample A1); or decyl oleate (e.g., Sample B1). However, replacement by coco caprylate may impact solubility of the mixture of crystalline sunscreen filters in certain embodiments (e.g., Sample B1). In embodiments, half of the dibutyl adipate may be replaced with diisobutyl adipate (e.g., Sample C₁) or diisopropyl adipate (e.g., Sample H1) but not isopropyl myristate, capric/caprylic triglyceride, or diethylhexyl maleate in certain embodiments (not shown). In embodiments, 10 wt. % of caprylic/capric triglyceride and 10 wt. % of dibutyl adipate may be replaced with 20 wt. % diisobutyl adipate (e.g., Sample N). Thus, for example, the ratio of butyloctyl salicylate to diisobutyl adipate may be 1:4.

In embodiments, the solvent may include butyloctyl salicylate, dibutyl adipate, caprylic/capric triglyceride, and diethylhexyl maleate. For example, dibutyl adipate, caprylic/capric triglyceride, and diethylhexyl maleate may be present in equal amounts (e.g., Sample T1). Thus, for example, butyloctyl salicylate, dibutyl adipate, caprylic/capric triglyceride, and diethylhexyl maleate may be present in a ratio of 1:1.3:1.3:1.3. In embodiments, butyloctyl salicylate may be excluded and the ratio of dibutyl adipate, caprylic/capric triglyceride, and diethylhexyl maleate may be 1:1:1 (e.g., Sample Z1). In embodiments, caprylic/capric triglyceride may be excluded and butyloctyl salicylate, dibutyl adipate, diethylhexyl maleate, and isopropyl myristate may be present in equal amounts (e.g., Sample P). Thus, for example the ratio of butyloctyl salicylate, dibutyl adipate, diethyl hexyl maleate, and isopropyl myristate may be 1:1:1:1. Similarly, caprylic/capric triglyceride may be excluded and butyloctyl salicylate, diisobutyl adipate, diethylhexyl maleate, and isopropyl myristate may be included in equal amounts (e.g., Sample Q).

Table 3 shows example sunscreen compositions having a synergistic combination of crystalline sunscreen filters. In embodiments, the example sunscreen compositions in Table 3 may be used in any formulation format. In one example, the example sunscreen compositions may be used in anhydrous spray formats as concentrates (e.g., SPF 30, 50, etc.).

TABLE 3

| | Diethylhexyl | | Bis- | Diethylamino | | | | |
|---|---|---|---|---|---|---|---|---|
| | Butamido | Butyl Methoxy- | ethylhexyloxyphenol | Hydroxybenzoyl | Polyhydroxystearic | | Behenyl | Total |
| Sample | Triazone | dibenzoylmethane | Methoxyphenyl | Hexyl Benzoate | Acid | Isododecane | Alcohol | Solvent |
| N2 | 5.00% | 5.00% | 3.00% | 1.00% | 0 | 10.00% | 0 | 40.00% |
| O2 | 5.00% | 5.00% | 3.00% | 0 | 0 | 5.00% | 0 | 39.00% |
| P2 | 6.00% | 4.50% | 3.00% | 2.00% | 0 | 10.00% | 0 | 30.00% |
| Q2 | 6.00% | 4.50% | 3.00% | 2.00% | 0 | 5.00% | 0 | 40.00% |
| R2 | 6.00% | 4.50% | 3.00% | 2.00% | 0 | 5.00% | 1.00% | 40.00% |
| S2 | 5.00% | 5.00% | 1.50% | 0 | 2.00% | 0 | 0 | 37.00% |
| T2 | 4.00% | 5.00% | 2.00% | 0 | 9.00% | 0 | 0 | 32.00% |
| U2 | 4.00% | 5.00% | 2.00% | 0 | 2.00% | 0 | 0 | 35.50% |
| V2 | 5.00% | 5.00% | 1.50% | 0 | 5.00% | 0 | 0 | 31.00% |

Table 3 shows various example formulations having different combinations of 10 crystalline sunscreen filters and solvent ingredients. Total solvent was mixed with ethanol present in an amount from 30 wt. % to 50 wt. %. As shown in Table 3, Sample N2 has a total solvent content of 40.0 wt. % consisting of butyloctyl salicylate, diethylhexyl naphthalate, capric/caprylic triglycerides, and butyl oleate each present in an amount of 5.0 wt. % to 10.0 wt. %. In addition, the cosmetically acceptable isoparaffinic carrier isododecane is used. Sample N2 passed multi-day freeze/thaw and multi-week cooling solubility evaluations. In addition, sample N2 exhibited very good water contact angle.

As shown in Table 3, Sample 02 has a total solvent content of 39.0 wt. % consisting of butyloctyl salicylate, isopropyl myristate, diisobutyl adipate, diethylhexyl maleate, diethylhexyl naphthalate, capric/caprylic triglycerides, and butyl oleate each present in an amount of 4.0 wt. % to 10.0 wt. %. In addition, the cosmetically acceptable isoparaffinic carrier isododecane is used. Sample 02 passed multi-day freeze/thaw and multi-week cooling solubility evaluations. Meanwhile, Sample P2 has a total solvent content of 30.0 wt. % consisting of butyloctyl salicylate, diethylhexyl naphthalate, capric/caprylic triglycerides, and butyl oleate each present in an amount of 5.0 wt. % to 10.0 wt. %. In addition, the cosmetically acceptable isoparaffinic carrier isododecane is used. Sample P2 passed multi-day freeze/thaw and multi-week cooling solubility evaluations. Notably, Sample P2 also includes about 2.0 wt. % acrylates/octylacrylamide copolymer.

As shown in Table 3, Samples Q2 and R2 each have a total solvent content of 40.0 wt. % consisting of butyloctyl salicylate, isopropyl myristate, diisobutyl adipate, diethylhexyl maleate, diethylhexyl naphthalate, capric/caprylic triglycerides, and butyl oleate each present in an amount of 5.0 wt. % to 10.0 wt. %. In addition, the cosmetically acceptable isoparaffinic carrier isododecane is used. Samples Q2 and R2 each passed multi-day freeze/thaw and multi-week cooling solubility evaluations. Notably, Samples Q2 and R2 included about 2.0 wt. % acrylates/octylacrylamide copolymer. In addition, Sample R2 included behenyl alcohol.

As shown in Table 3, Sample S2 has a total solvent content of 37.0 wt. % consisting of butyloctyl salicylate, diisopropyl adipate, isopropyl myristate, diisobutyl adipate, diethylhexyl maleate, and octyl palmitate each present in an amount of 2.0 wt. % to 10.0 wt. %. Sample S2 passed multi-day freeze/thaw and multi-week cooling solubility evaluations. In addition, Sample S2 exhibited excellent contact angle. Similar to Sample S2, samples T2, U2, and V2 having varying levels of polyhydroxystearic acid also passed multi cycle freeze/thaw evaluations. In addition to other aspects presented herein, polyhydroxystearic acid may surprisingly be used as a solvent. In this regard, butyl methoxydibenzoylmethane tested at 1.0 wt. % to 20.0 wt. % passed four freeze-thaw cycles at 10 wt. % and bis-ethylhexyloxyphenol methoxyphenyl triazine tested at 1.0 wt. % to 20.0 wt. % passed one freeze-thaw cycle at 11 wt. % to 13.0 wt. %, while the combination of the two passed four freeze-thaw cycles when each were present at 10.0 wt. %. However, changes to viscosity/appearance with no formed crystals after four freeze-thaw cycles occurred when butyl methoxydibenzoylmethane and bis-ethylhexyloxyphenol methoxyphenyl triazine were present at 0.5 wt. % and 5.5 wt. %, respectively. Butyl methoxydibenzoylmethane and bis-ethylhexyloxyphenol methoxyphenyl triazine were also stable under 24-hour cooling conditions when present from 1.0 wt. % to 14.0 wt. % and 1.0 wt. % to 7.0 wt. %, respectively.

Accordingly, in various embodiments, a ratio of butyloctyl salicylate, isopropyl myristate, diisobutyl adipate, butyl oleate, diethylhexyl maleate, diethylhexyl 2,6-naphthalate, and capric/caprylic triglyceride may be 1:1:1:1:1:1:2. Additionally or alternatively, in various embodiments, a ratio of diethylhexyl butamido triazone, bis-ethylhexyloxyphenol methoxyphenyl triazine, butyl methoxydibenzoylmethane, and diethylamino hydroxybenzoyl hexyl benzoate may be 1:1.25:0.5:0.3 to 1:2:1.3:1. Diethylamino hydroxybenzoyl hexyl benzoate may be increased without disrupting solubility. A ratio of crystalline filters to solvent may be, for example, 1:1 to 1:4, preferably 1:1.9 to 1:3.2.

Additional anhydrous sunscreen compositions, in concentrate and/or aerosol formats, may include butyl methoxydibenzoylmethane present in an amount of 0 wt. % to 5.0 wt. %, preferably 3.0 wt. % to 5.0 wt. % when present, diethylhexyl butamido triazone present in an amount of 0 wt. % to 6.0 wt. %, preferably 1.0 wt. % to 6.0 wt. % when present, diethylamino hydroxybenzoyl hexyl benzoate present in an amount of 0 wt. % to 5.0 wt. %, preferably 1.0 wt. % to 3.0 wt. % when present, and bis-ethylhexyloxyphenol methoxyphenyl triazine present in an amount of 0 wt. % to 4.0 wt. %, preferably about 3.0 wt. % when present, based on the total weight of the sunscreen composition. In one example, the ratio of butyl methoxydibenzoylmethane, diethylhexyl butamido triazone, diethylamino hydroxybenzoyl hexyl benzoate, and bis-ethylhexyloxyphenol methoxyphenyl triazine is 1.3:1:3:2.

In embodiments, butyl methoxydibenzoylmethane may improve the solubility of bis-ethylhexyloxyphenol methoxyphenyl triazine. For example, about 5.0 wt. % of butyl methoxydibenzoylmethane may be preferable to improve the solubility of about 3.0 wt. % of bis-ethylhexyloxyphenol methoxyphenyl triazine. However, bis-ethylhexyloxyphenol methoxyphenyl triazine may be optional and therefore excluded without substantially effecting to the solubility of the remaining crystalline sunscreen filters. Meanwhile, reducing from about 6% diethylhexyl butamido triazone does negatively impact solubility at least of butyl methoxydibenzoylmethane and bis-ethylhexyloxyphenol methoxyphenyl triazine. Diethylhexyl butamido triazone may be reduced with the reduction of bis-ethylhexyloxyphenol methoxyphenyl triazine, indicating that diethylhexyl butamido triazone also helps keep bis-ethylhexyloxyphenol methoxyphenyl triazine in solution. Also, the addition of about 1.0 wt. % to about 2.0 wt. % diethylamino hydroxybenzoyl hexyl benzoate may similarly improve solubility of crystalline sunscreen filters such as butyl methoxydibenzoylmethane in the presence of about 6.0 wt. % diethylhexyl butamido triazone, although also optional.

Notably, solvent having butyloctyl salicylate, capric/caprylic triglyceride, diisobutyl adipate, diethylhexyl maleate, butyl maleate, butyl oleate, and isopropyl myristate surprisingly takes 35 g to solubilize 15.5 g of butyl methoxydibenzoylmethane, diethylhexyl butamido triazone, diethylamino hydroxybenzoyl hexyl, and bis-ethylhexyloxyphenol methoxyphenyl triazine. This is unexpected since it theoretically takes at least 37 g of the solvent combination to solvate 3 g of bis-ethylhexyloxyphenol methoxyphenyl triazine (a filter that is harder to solubilize than even avobenzone) and 42.8 g of the solvent combination to solvate 15.5 g of the crystalline filter combination. Thus, solubilizers such 2-ethylhexyl alpha-cyano-beta-phenylcinnamate (octocrylene), 3,3,5-trimethylcyclohexyl salicylate (homosalate), octyl salicylate (octisalate), ethylhexyl benzoate, phenethyl benzoate, and/or C₁₂-C₁₅ alkyl benzoate may intentionally be omitted to address concerns while surprisingly solubilizing relatively more crystalline sunscreen filters with relatively less solvent content.

In embodiments, caprylic/capric triglyceride may be replaced by diisopropyl adipate, dibutyl adipate, or combinations thereof. For example, 10 wt. % of caprylic/capric triglyceride may be replaced by diisopropyl adipate or dibutyl adipate. Meanwhile, diisobutyl adipate may be replaced with diisopropyl adipate. For example, 5 wt. % of diisobutyl adipate may be replaced by diisopropyl adipate. However, removal of butyloctyl salicylate may allow for crystal formation. Multiple other solvent combinations are also possible. Notable solvents for stability include, without limitation, butyloctyl salicylate, isopropyl myristate, diethylhexyl maleate, and butyl oleate which when any are removed may render a formulation unstable in crystal evaluations. Moreover, the inclusion of behenyl alcohol for anti-migration may benefit from additional solvent as illustrated with reference to Sample S2.

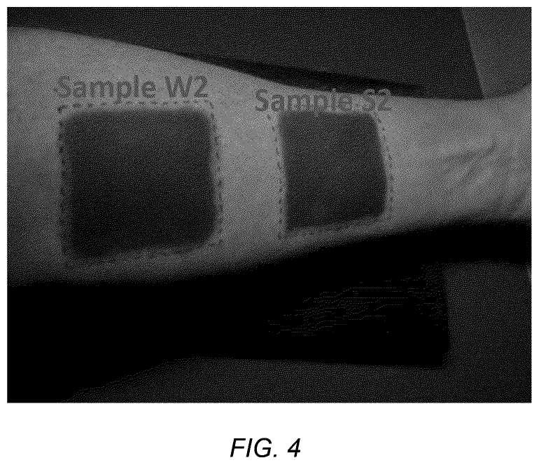

Turning now to FIG. 2, Sample R2 was compared to an example sunscreen composition formulated to be the same as Sample R2 except without behenyl alcohol (Sample W2). As shown in FIG. 2, there is a marked difference in migration after a 15-minute dry time and five minutes water emersion between alcohol-based sunscreen compositions having film former (e.g., acrylates/octylacrylamide copolymer), crystalline sunscreen filters as shown in Table 3, and solvent that includes behenyl alcohol (Sample S2) compared to solvent without behenyl alcohol (Sample W2).

Notably, embodiments of alcohol-based sunscreen compositions having behenyl alcohol are also stable without precipitates at ambient temperature. This is surprising, since behenyl alcohol is relatively less soluble in ethanol and since relatively longer alkyl chain alcohols are believed to increase the risk of precipitation or precipitation with crystalline sunscreen filters. The use of behenyl alcohol also provides unexpected benefits compared to other carriers such as cetearyl alcohol, cetyl alcohol, stearyl alcohol, ozokerite, and paraffin that do not achieve the same performance and in various cases exhibited recrystallization of sunscreen filters in alcohol-based compositions. Also, propellant used in aerosol spray formats do not cause precipitation of behenyl alcohol or of crystalline sunscreen filters.

Additionally, film former (e.g., acrylates/octylacrylamide copolymer, VA/butyl maleate/isobornyl acrylate copolymer, etc.) and a fatty alcohol (e.g., behenyl alcohol) provides a marked reduction in migration of solvent and/or of crystalline sunscreen filter in alcohol sunscreen compositions. Preferably, the sunscreen composition includes acrylates/octylacrylamide copolymer and behenyl alcohol. Additionally or alternatively, a film remaining after volatile carrier evaporates can provide benefits such as reliable photoprotection, good water resistance (e.g., 40 minutes, 80 minutes, good contact angles, etc.), and/or good sensory attributes.

Table 4 shows example sunscreen compositions having a synergistic combination of crystalline sunscreen filter(s) and/or solvent(s) according to embodiments. In embodiments, the example sunscreen compositions in Table 4 may be used in any formulation format. In one example, the example sunscreen compositions may be used in O/W lotions to provide an SPF of 15, 30, and 50.

TABLE 4

| Component (INCI) | Description | Approx. wt. % |
|---|---|---|
| Example Main Ingredients | | |
| Diethylhexyl Butamido Triazone | Sunscreen Filter | 2.0-5.0 |
| | (crystalline) | |
| Butyl Methoxydibenzoylmethane | Sunscreen Filter | 3.0-4.5 |
| | (crystalline) | |
| Bis-Ethylhexyloxyphenol Methoxyphenyl | Sunscreen Filter | 1.0-3.0 |
| Triazine | (crystalline) | |
| Diethylamino Hydroxybenzoyl Hexyl Benzoate | Sunscreen Filter | 0.5-3.0 |
| | (crystalline) | |
| Butyloctyl Salicylate, Caprylic/Capric | Solvent (Ester) | 15.0-25.0 |
| Triglyceride, Dibutyl Adipate, Diisobutyl | | |
| Adipate | | |
| Additional Ingredients | | |
| Water, Disodium EDTA, Sodium Hydroxide, | Solvent (aqueous), | Q.S. 100 |
| Phenoxyethanol, Chlorphenesin, Caprylyl | Chelating agent, pH | |
| Glycol, Acrylates/C₁₀-C₃₀ Alkyl Acrylate | adjuster, | |
| Crosspolymer, Xanthan Gum, Glycerin, | Preservative system, | |
| Propanediol, Panthenol, Cetearyl Alcohol (and) | Rheology modifier, | |
| Dicetyl Phosphate (and) Ceteth-10 Phosphate, | Thickener, Skin | |
| Cetearyl Alcohol (and) Coco-Glucoside, | conditioner, | |
| Acrylates/C₁₂-C₂₂ Alkyl Methacrylate | Emulsifier, Film | |
| Copolymer, Sodium Ascorbyl Phosphate, | Former, Extract, | |
| Tocopheryl Acetate, Butter (Mango, Shea), | Vitamin, Colorant, | |
| Aloe, Mica, Iron Oxides, Titanium Dioxide, | Fragrance, Dispersant | |
| Starch (Rice), Fragrance, Alcohol Denat. (if | | |
| present, ≤1.0 wt. %). | | |

Personal care compositions were formulated with the main ingredients at the levels shown in Table 4. Any or all of the listed additional ingredients were added in a sufficient amount to bring the total amount of the sunscreen composition up to 100 wt. %. For example, various example formulations include all of the additional ingredients shown in Table 4 except with or without butter, colorant, fragrance, and/or alcohol. In addition, various example formulations included variations in the amount of ingredients. For example, the levels of crystalline sunscreen filter(s) varied to provide a desired SPF or solubility profile. Similarly, the levels of solvent(s) varied to provide a desired solubility or sensory profile.

Table 5 shows example sunscreen compositions having a synergistic combination of crystalline sunscreen filter(s) and/or solvent(s) according to embodiments. In embodiments, the example sunscreen compositions in Table 5 may be used in any formulation format. In one example, the example sunscreen compositions may be used in anhydrous spray formats as concentrates to provide an SPF of SPF of 15, 30, and 50.

TABLE 5

| Component (INCI) | Description | Approx. wt. % |
|---|---|---|
| Example Main Ingredients | | |
| Diethylhexyl Butamido Triazone | Sunscreen Filter | 2.5-6.0 |
| | (crystalline) | |
| Butyl Methoxydibenzoylmethane | Sunscreen Filter | 4.0-5.0 |
| | (crystalline) | |
| Bis-Ethylhexyloxyphenol Methoxyphenyl | Sunscreen Filter | 0-3.0 |
| Triazine | (crystalline) | |
| Diethylamino Hydroxybenzoyl Hexyl Benzoate | Sunscreen Filter | 0-6.0 |
| | (crystalline) | |
| Butyloctyl Salicylate, Caprylic/Capric | Solvent (Ester) | 20.0-40.0 |
| Triglyceride, Diisobutyl Adipate, Isopropyl | | |
| Myristate, Butyl Oleate, Diethylhexyl Maleate, | | |
| Diethylhexyl 2,6-Naphthalate | | |
| Additional Ingredients | | |
| Alcohol Denat., Behenyl Alcohol, | Solvent (volatile), | Q.S. 100 |
| Acrylates/Octylacrylamide Copolymer, | Migration Modifier | |
| Polyester-7 (and) Neopentyl Glycol | System, Film Former, | |
| Diheptanoate, Isododecane, Panthenol, | Dispersant, Skin | |
| Extract (Fruit), Ascorbyl Palmitate, Tocopheryl | Conditioner, Extract, | |
| Acetate, Fragrance, Water (if present, ≤1.0 wt. | Vitamin, Fragrance | |
| %). | | |

Personal care compositions were formulated with the main ingredients at the levels shown in Table 5. Any or all of the listed additional ingredients were added in a sufficient amount to bring the total amount of the sunscreen composition up to 100 wt. %. For example, various example formulations include all of the additional ingredients except with or without behenyl alcohol, acrylates/octylacrylamide copolymer, and/or polyester-7 (and) neopentyl glycol diheptanoate. When present, these ingredients were added in the amounts described herein. The levels of crystalline sunscreen filter(s) also varied to provide a desired SPF or solubility profile. Similarly, the levels of solvent(s) varied to provide a desired solubility or sensory profile. It is understood that when a concentrate is to be aerosolized, levels of ingredients in the concentrate can be determined to account for a final concentration of the ingredients in the spray format and therefore the levels of the ingredients present in the aerosolized spay product are also within the scope of various aspects of embodiments.

It is also understood that the examples above include aspects of non-limiting embodiments, and that listed ingredients or respective levels or ratios may be modified based on various formulation criteria such as, without limitation, format, photoprotection, sensory profile, water resistance, diffusion profile, solubility profile, and so on.

### Additional Notes and Examples

Additional Examples include a sunscreen composition, a personal care product Including the personal care composition, a method of making the personal care composition and/or the personal care product, a method of using the personal care composition and/or the personal care product (a consumer packaged product), or combinations thereof.

Additional Example 1 may include a sunscreen composition comprising a solvent including an ester and a sunscreen filter including a crystalline ultraviolet filter.

Additional Example 2 may include the sunscreen composition of Additional Example 1, comprising a solvent including an ester and a mixture of sunscreen filters dissolved in the solvent, wherein the mixture includes a crystalline ultraviolet A (UVA) filter and a crystalline UVB filter, wherein the ratio of the crystalline UVA filter to the crystalline UVB filter is about 3:1 to about 1:5.

Additional Example 3 may include the sunscreen composition of any of Additional Examples 1-2, wherein the crystalline UVA filter includes butyl methoxydibenzoylmethane and excludes diethylamino hydroxybenzoyl hexyl benzoate, and wherein the crystalline UVB filter includes diethylhexyl butamido triazone.

Additional Example 4 may include the sunscreen composition of any of Additional Examples 1-3, wherein the ratio of butyl methoxydibenzoylmethane to diethylhexyl butamido triazone is 1.25:1 to 1:1.25.

Additional Example 5 may include the sunscreen composition of any of Additional Examples 1-4, wherein butyl methoxydibenzoylmethane is present in an amount of about 4 wt. % to about 5 wt. %, based on the total weight of the sunscreen composition, and wherein diethylhexyl butamido triazone is present in an amount of about 4 wt. % to about 6 wt. %, based on the total weight of the sunscreen composition.

Additional Example 6 may include the sunscreen composition of any of Additional Examples 1-5, wherein the crystalline UVB filter includes ethylhexyl triazone.

Additional Example 7 may include the sunscreen composition of any of Additional Examples 1-6, wherein diethylhexyl butamido triazone is present in an amount ≥80% of the amount of butyl methoxydibenzoylmethane present in the sunscreen composition, wherein diethylhexyl butamido triazone is present in an amount ≥80% of the total amount of all crystalline UVB sunscreen filters present in the sunscreen composition, or combinations thereof.

Additional Example 8 may include the sunscreen composition of any of Additional Examples 1-7, wherein ethylhexyl triazone is present in an amount less than the amount of diethylhexyl butamido triazone present in the sunscreen composition.

Additional Example 9 may include the sunscreen composition of any of Additional Examples 1-8, wherein the crystalline UVA filter includes diethylamino hydroxybenzoyl hexyl benzoate, and wherein the crystalline UVB filter includes diethylhexyl butamido triazone, ethylhexyl triazone, or combinations thereof.

Additional Example 10 may include the sunscreen composition of any of Additional Examples 1-9, wherein the ratio of diethylamino hydroxybenzoyl hexyl benzoate to diethylhexyl butamido triazone or to ethylhexyl triazone is from 1:1 to 1:5.

Additional Example 11 may include the sunscreen composition of any of Additional Examples 1-10, wherein diethylamino hydroxybenzoyl hexyl benzoate is present in an amount of about 1 wt. % to about 5 wt. %, based on the total weight of the sunscreen composition, and wherein diethylhexyl butamido triazone is present in an amount of about 1 wt. % to about 10 wt. %, based on the total weight of the sunscreen composition.

Additional Example 12 may include the sunscreen composition of any of Additional Examples 1-11, wherein diethylamino hydroxybenzoyl hexyl benzoate is present in an amount of about 1 wt. % to about 5 wt. %, based on the total weight of the sunscreen composition, and wherein ethylhexyl triazone is present in an amount of about 1 wt. % to about 5 wt. %, based on the total weight of the sunscreen composition.

Additional Example 13 may include the sunscreen composition of any of Additional Examples 1-12, wherein the crystalline UVA filter further includes butyl methoxydibenzoylmethane.

Additional Example 14 may include the sunscreen composition of any of Additional Examples 1-13, wherein the crystalline ultraviolet filter and/or the mixture of sunscreen filters includes a crystalline UVA/UVB filter.

Additional Example 15 may include the sunscreen composition of any of Additional Examples 1-14, wherein the crystalline UVA/UVB filter includes bis-ethylhexyloxyphenol methoxyphenyl triazine.

Additional Example 16 may include the sunscreen composition of any of Additional Examples 1-15, wherein bis-ethylhexyloxyphenol methoxyphenyl triazine is present in an amount of up to about 3 wt. %, based on the total weight of the composition.

Additional Example 17 may include the sunscreen composition of any of Additional Examples 1-16, wherein the solvent includes an ester of salicylic acid, an ester of adipic acid, an ester of maleic acid, an ester of neopentanoic acid, an ester of C₈-C₁₈ fatty acids, or combinations thereof.

Additional Example 18 may include the sunscreen composition of any of Additional Examples 1-17, wherein the solvent includes butyloctyl salicylate, diisobutyl adipate, dibutyl adipate, diisopropyl adipate, diethylhexyl adipate, diethylhexyl maleate, isostearyl neopentanoate, butyl oleate, decyl oleate, caprylic/capric triglyceride, isopropyl myristate, octyl palmitate, coco caprylate, or combinations thereof.

Additional Example 19 may include the sunscreen composition of any of Additional Examples 1-18, wherein the solvent includes butyloctyl salicylate, dibutyl adipate, diisobutyl adipate, and caprylic/capric triglyceride.

Additional Example 20 may include the sunscreen composition of any of Additional Examples 1-19, wherein butyloctyl salicylate is present in an amount of 4 wt. % to 9 wt. %, dibutyl adipate is present in an amount of 4 wt. % to 13 wt. %, diisobutyl adipate is present in an amount of 2 wt. % to 20 wt. %, and caprylic/capric triglyceride is present in an amount of about 5 wt. % to about 10 wt. %, based on the total weight of the composition.

Additional Example 21 may include the sunscreen composition of any of Additional Examples 1-20, wherein the solvent includes one or more adipates present in a total amount of about 8 wt. % to about 10 wt. %, caprylic/capric triglyceride present in an amount of about 8 wt. % to about 10 wt. %, and butyl octyl salicylate in an amount of 0 wt. % to about 5.0 wt. %, wherein the ratio of adipates to caprylic/capric triglyceride is about 1:1, excludes diisobutyl adipate and includes butyloctyl salicylate, excludes caprylic/capric triglyceride and includes butyl oleate, dimethyl isosorbide, diethylhexyl adipate, isostearyl neopentanoate, decyl oleate, diethylhexyl maleate, coco caprylate, or combinations thereof, excludes one or more of dibutyl adipate or coco caprylate and includes butyl oleate, dimethyl isosorbide, diethylhexyl adipate, isostearyl neopentonate, decyl oleate, or combinations thereof, includes dibutyl adipate and one of diisobutyl adipate or diisopropyl adipate in a ratio of 1:1, and excludes isopropyl myristate, caprylic/capric triglyceride, or diethylhexyl maleate, excludes dibutyl adipate and caprylic/capric triglyceride and includes dibutyl adipate in an amount greater than zero and ≤20 wt. %, based on the total weight of the composition, includes butyloctyl salicylate, dibutyl adipate, caprylic/capric triglyceride, and diethylhexyl maleate, excludes caprylic/capric triglyceride and includes butyloctyl salicylate, dibutyl adipate, diethylhexyl maleate, and isopropyl myristate, or excludes butyloctyl salicylate and include dibutyl adipate, caprylic/capric triglyceride, and diethyl hexyl maleate.

Additional Example 22 may include the sunscreen composition of any of Additional Examples 1-21, wherein the solvent further includes dimethyl isosorbide.

Additional Example 23 may include the sunscreen composition of any of Additional Examples 1-22, wherein the solvent excludes ethylhexyl benzoate, phenethyl benzoate, C₁₂-C₁₅ alkyl benzoate, or combinations thereof.

Additional Example 24 may include the sunscreen composition of any of Additional Examples 1-23, wherein the solvent is present in a total amount of 25.0 wt. % to 40.0 wt. %, based on the total weight of the composition.

Additional Example 25 may include the sunscreen composition of any of Additional Examples 1-24, wherein the sunscreen composition is in the form of a gel, a lotion, a cream, a spray, a mist, a stick, a mousse, a foam, an ointment, a milk, a base, a concentrate, a mist, or combinations thereof.

Additional Example 26 may include the sunscreen composition of any of Additional Examples 1-25, wherein the sunscreen composition is an aqueous emulsion, and wherein the solvent is part of an oil phase of the aqueous emulsion.

Additional Example 27 may include the sunscreen composition of any of Additional Examples 1-26, wherein the sunscreen composition is a substantially anhydrous composition and includes a C₂-C₄ alcohol present in an amount of about 30 wt. % to about 70 wt. %, based on the total weight of the composition.

Additional Example 28 may include the sunscreen composition of any of Additional Examples 1-27, further including a fatty alcohol.

Additional Example 29 may include the sunscreen composition of any of Additional Examples 1-28, wherein the fatty alcohol includes behenyl alcohol present in an amount of about 0.1 wt. % to about 1.0 wt. %, based on the total weight of the composition.

Additional Example 30 may include the sunscreen composition of any of Additional Examples 1-29, further including a film former.

Additional Example 31 may include the sunscreen composition of any of Additional Examples 1-30, wherein the film former includes a (meth)acrylate acid copolymer.

Additional Example 32 may include the sunscreen composition of any of Additional Examples 1-31, wherein the (meth)acrylate acid copolymer includes acrylates/octylacrylamide copolymer present in an amount of about 1.0 wt. % to about 2.0 wt. %, based on the total weight of the composition.

Additional Example 33 may include the sunscreen composition of any of Additional Examples 1-32, further including an isoparaffin, an organosilicone, an ester of naphthalene dicarboxylic acid, an emulsifier, or combinations thereof.

Additional Example 34 may include the sunscreen composition of any of Additional Examples 1-33, including isododecane, isohexadacane, disiloxane, dimethicone, cyclopentasiloxane, diethylhexyl 2,6-naphthalate, polyhydroxystearic acid, or combinations thereof.

Additional Example 35 may include the sunscreen composition of any of Additional Examples 1-34, wherein the solvent includes butyloctyl salicylate, isopropyl myristate, diisobutyl adipate, butyl oleate, diethylhexyl maleate, diethylhexyl 2,6-naphthalate, and capric/caprylic triglyceride in a ratio of 1:1:1:1:1:1:2.

Additional Example 36 may include the sunscreen composition of any of Additional Examples 1-35, including diethylhexyl butamido triazone, bis-ethylhexyloxyphenol methoxyphenyl triazine, butyl methoxydibenzoylmethane, and diethylamino hydroxybenzoyl hexyl benzoate in a ratio of 1:1.25:0.5:0.3 to 1:2:1.3:1.

Additional Example 37 may include the sunscreen composition of any of Additional Examples 1-36, including diethylhexyl butamido triazone, bis-ethylhexyloxyphenol methoxyphenyl triazine, butyl methoxydibenzoylmethane, and diethylamino hydroxybenzoyl hexyl benzoate in a ratio of 1:1.8:0:0.

Additional Example 38 may include the sunscreen composition of any of Additional Examples 1-37, wherein the sunscreen composition further includes a propellant.

Additional Example 39 may include the sunscreen composition of any of Additional Examples 1-38, wherein the propellant includes isobutane, butane, propane, dimethyl ether, methyl ether, or combinations thereof.

Additional Example 40 may include the sunscreen composition of any of Additional Examples 1-39, wherein the mixture of sunscreen filters includes a sunscreen filter selected from a p-aminobenzoic acid, a benzophenone, a camphor, a cinnamate, a dibenzoylmethane, a salicylate, an imidazole, a triazole, a triazine, a triazole, triazone, a metal oxide, or combinations thereof.

Additional Example 41 may include the sunscreen composition of any of Additional Examples 1-40, wherein the sunscreen composition excludes benzophenone-3, octyl methoxycinnamate, 2-ethylhexyl-2-cyano-3,3-diphenylacrylate, 3,3,5-trimethylcyclohexyl salicylate, and 2-ethylhexyl salicylate.

Additional Example 42 may include the sunscreen composition of any of Additional Examples 1-41, wherein the solvent includes an ester of salicylic acid, an ester of adipic acid, an ester of maleic acid, an ester of neopentanoic acid, an ester of C₈-18 fatty acids, or combinations thereof, and wherein the crystalline ultraviolet filter includes bis-ethylhexyloxyphenol methoxyphenyl triazine.

Additional Example 43 may include the sunscreen composition of any of Additional Examples 1-44, wherein the solvent includes butyloctyl salicylate, an adipate selected from dibutyl adipate and/or diisopropyl adipate, and capric/caprylic triglyceride, wherein the crystalline ultraviolet filter includes bis-ethylhexyloxyphenol methoxyphenyl triazine, and wherein the sunscreen composition excludes one or more of octocrylene, homosalate, octisalate, octinoxate, ethylhexyl benzoate, phenethyl benzoate, or C₁₂-C₁₅ alkyl benzoate.

Additional Example 45 may include the sunscreen composition of any of Additional Examples 1-44, wherein the solvent includes butyloctyl salicylate, capric/caprylic triglyceride, diisobutyl adipate, diethylhexyl maleate, butyl maleate, butyl oleate, and isopropyl myristate, wherein the crystalline ultraviolet filter includes bis-ethylhexyloxyphenol methoxyphenyl triazine, and wherein the sunscreen composition excludes one or more of octocrylene, homosalate, octisalate, octinoxate, ethylhexyl benzoate, phenethyl benzoate, or C₁₂-C₁₅ alkyl benzoate.

Additional Example 44 may include the sunscreen composition of any of Additional Examples 1-43, wherein the crystalline ultraviolet filter includes bis-ethylhexyloxyphenol methoxyphenyl triazine and one or more of butyl methoxydibenzoylmethane, diethylhexyl butamido triazone, diethylamino hydroxybenzoyl hexyl benzoate, or ethylhexyl triazone.

Additional Example 45 may include the sunscreen composition of any of Additional Examples 1-44, wherein the crystalline ultraviolet filter includes a synergistic combination of bis-ethylhexyloxyphenol methoxyphenyl triazine and butyl methoxydibenzoylmethane.

Additional Example 46 may include the sunscreen composition of any of Additional Examples 1-45, wherein the crystalline ultraviolet filter includes a synergistic combination of bis-ethylhexyloxyphenol methoxyphenyl triazine and diethylhexyl butamido triazone.

Additional Example 47 may include the sunscreen composition of any of Additional Examples 1-46, wherein the sunscreen composition includes a silicone selected from a polyalkylsiloxane, an alkyl substituted dimethicone, a dimethiconol, a polyalkylaryl siloxane, or combinations thereof, and wherein the sunscreen composition further includes a silicone emulsifier, a silicone elastomer gel agent, or combinations thereof.

Additional Example 48 may include the sunscreen composition of any of Additional Examples 1-47, wherein the sunscreen composition further includes one or more film formers, water, surfactants, emulsifiers, alcohols, rheology agents, skin conditioners, waxes, elastomers, sterols, amino acids, colorants, dyes, pigments, powders, pH adjusters, fragrances, vitamins, preservatives, preservative boosters, foaming agents, chelating agents, cleansers, stabilizers, dispersants, essential fatty acids, sphingolipids, self-tanning compounds, fillers, antioxidants, minerals, keratolytics, retinoids, hormonal compounds, anti-aging agents, alpha-keto acids, anti-microbial agents, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin pollution protectants, skin penetration enhancers, exfoliants, lubricants, staining agents, depigmenting agents, hypopigmenting agents, extracts, propellants, or combinations thereof.

Additional Example 49 may include a method to manufacture the sunscreen composition of any of Additional Examples 1-48, a personal care product including the sunscreen composition of any of Additional Examples 1-48, or combinations thereof.

Additional Example 50 may include the method of Additional Example 50, comprising mixing two or more ingredients of the sunscreen composition of any of Additional Examples 1-48, combining the sunscreen composition of any of Additional Examples 1-48 with a product package, or combinations thereof.

Additional Example 51 may include a method to use the sunscreen composition of any of Additional Examples 1-48, a personal care product including the sunscreen composition of any of Additional Examples 1-48, or combinations thereof.

Additional Example 52 may include the method of Additional Example 51, comprising applying the sunscreen composition of any of Additional Examples 1-48 onto a keratinous or hirsute substrate.

Additional Example 53 may include the method of any of Additional Examples 51-52, comprising applying the sunscreen composition from the personal care product.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

Accordingly, while the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

1-20. (canceled)

21. A sunscreen composition comprising:
a solvent including an ester, wherein the ester includes an ester of salicylic acid and an ester of adipic acid, wherein the ester of salicylic acid includes butyloctyl salicylate and the ester of adipic acid includes diisopropyl adipate, wherein butyloctyl salicylate is present in an amount of about 4 wt. % to about 9 wt. %, based on the total weight of the sunscreen composition, and wherein diisopropyl adipate is present in an amount of about 5 wt. % to about 10 wt. %, based on the total weight of the sunscreen composition; and
a mixture of sunscreen filters dissolved in the solvent, wherein the mixture includes:
a crystalline ultraviolet A (UVA) filter; and
a crystalline UVB filter, wherein the ratio of the crystalline UVA filter to the crystalline UVB filter is about 3:1 to about 1:5.

22. The sunscreen composition of claim 21, wherein the crystalline UVA filter includes butyl methoxydibenzoylmethane and excludes diethylamino hydroxybenzoyl hexyl benzoate, wherein butyl methoxydibenzoylmethane is present in an amount of 3.0 wt. % to 5.0 wt. %, based on the total weight of the sunscreen composition, and wherein the crystalline UVB filter includes diethylhexyl butamido triazone.

23. The sunscreen composition of claim 22, wherein the crystalline UVB filter further includes ethylhexyl triazone.

24. The sunscreen composition of claim 21, wherein the crystalline UVA filter includes diethylamino hydroxybenzoyl hexyl benzoate, wherein diethylamino hydroxybenzoyl hexyl benzoate is present in an amount of 1.0 wt. % to 5.0 wt. %, based on the total weight of the sunscreen composition, and wherein the crystalline UVB filter includes diethylhexyl butamido triazone, ethylhexyl triazone, or combinations thereof.

25. The sunscreen composition of claim 24, wherein the ratio of diethylamino hydroxybenzoyl hexyl benzoate to diethylhexyl butamido triazone or to ethylhexyl triazone is from 1:1 to 1:5.

26. The sunscreen composition of claim 24, wherein the crystalline UVA filter further includes butyl methoxydibenzoylmethane.

27. The sunscreen composition of claim 21, wherein the mixture of sunscreen filters further includes bis-ethylhexyloxyphenol methoxyphenyl triazine present in an amount of up to about 3 wt. %, based on the total weight of the composition.

28. The sunscreen composition of claim 21, wherein the solvent further includes an ester of maleic acid, an ester of neopentanoic acid, an ester of C₈-C₁₈ fatty acids, or combinations thereof.

29. The sunscreen composition of claim 28, wherein the solvent further includes bdiisobutyl adipate, dibutyl adipate, diethylhexyl adipate, diethylhexyl maleate, isostearyl neopentanoate, butyl oleate, decyl oleate, caprylic/capric triglyceride, isopropyl myristate, octyl palmitate, coco caprylate, or combinations thereof.

30. The sunscreen composition of claim 28, wherein the solvent further includes dimethyl isosorbide.

31. The sunscreen composition of claim 28, wherein the solvent excludes ethylhexyl benzoate, phenethyl benzoate, C₁₂-C₁₅ alkyl benzoate, or combinations thereof.

32. The sunscreen composition of claim 28, wherein the solvent is present in a total amount of 25.0 wt. % to 40.0 wt. %, based on the total weight of the composition.

33. The sunscreen composition of claim 28, wherein the sunscreen composition is an aqueous emulsion, and wherein the solvent is part of an oil phase of the aqueous emulsion.

34. The sunscreen composition of claim 28, wherein the sunscreen composition is a substantially anhydrous composition and includes a C₂-C₄ alcohol present in an amount of about 30 wt. % to about 70 wt. %, based on the total weight of the composition.

35. The sunscreen composition of claim 34, further comprising a fatty alcohol including behenyl alcohol present in an amount of about 0.1 wt. % to about 1.0 wt. %, based on the total weight of the composition.

36. The sunscreen composition of claim 34, further comprising a (meth)acrylate acid copolymer including acrylates/octylacrylamide copolymer present in an amount of about 1.0 wt. % to about 2.0 wt. %, based on the total weight of the composition.

37. The sunscreen composition of claim 34, further including an isoparaffin, an organosilicone, an ester of naphthalene dicarboxylic acid, an emulsifier, or combinations thereof.

38. The sunscreen composition of claim 37, including isododecane, isohexadacane, disiloxane, dimethicone, cyclopentasiloxane, diethylhexyl 2,6-naphthalate, polyhydroxystearic acid, or combinations thereof.

39. The sunscreen composition of claim 21, further comprising a fatty alcohol including behenyl alcohol present in an amount of about 0.1 wt. % to about 1.0 wt. %, based on the total weight of the composition.

40. The sunscreen composition of claim 21, wherein the sunscreen composition excludes benzophenone-3, octyl methoxycinnamate, 2-ethylhexyl-2-cyano-3,3-diphenylacrylate, 3,3,5-trimethylcyclohexyl salicylate, 2-ethylhexyl salicylate, or combinations thereof.
